(19) United States
(12) Patent Application Publication
(10) Pub. No.: US 2024/0076553 A1
(43) Pub. Date: Mar. 7, 2024

(54) NONIONIC, REDOX-CLEAVABLE SURFACTANT FOR MASS SPECTROMETRY-BASED PROTEOMICS AND STRUCTURAL BIOLOGICAL APPLICATIONS

(71) Applicant: Wisconsin Alumni Research Foundation, Madison, WI (US)

(72) Inventors: Kyle BROWN, Madison, WI (US); Song JIN, Madison, WI (US); Ying GE, Madison, WI (US); Morgan GUGGER, Champaign, IL (US)

(21) Appl. No.: 18/451,614

(22) Filed: Aug. 17, 2023

Related U.S. Application Data
(60) Provisional application No. 63/399,004, filed on Aug. 18, 2022.

(51) Int. Cl. C09K 23/32; C07H 5/10; G01N 33/68

(52) U.S. Cl. CPC C09K 23/32; C07H 5/10; G01N 33/6848; G01N 2570/00

(57) ABSTRACT

The present invention provides nonionic cleavable surfactants (NCS), specifically n-Decyl-disulfide-β-D-maltoside (DSSM), suitable for MS-based proteomics and analysis. These surfactants are designed to mimic the properties of a commonly used surfactant in structural biology, n-dodecyl-β-d-maltoside (DDM), but contain a disulfide bond that allows for facile cleavage and surfactant removal before analysis. DSSM and other NCS are compatible with native mass spectrometry, top-down and bottom-up proteomics, ESI-MS and other analytical techniques, and reduce signal suppression typically observed with other surfactants. DSSM and other NCS provide versatile surfactants that can facilitate protein sample preparation under non-denaturing conditions for a myriad of proteomic and structural biology applications and act as a general replacement for DDM.

### CROSS-REFERENCE TO RELATED APPLICATION

This application claims priority from U.S. Provisional Application No. 63/399,004, filed Aug. 18, 2022, which is incorporated by reference herein to the extent that there is no inconsistency with the present disclosure.

### STATEMENT REGARDING FEDERALLY SPONSORED RESEARCH OR DEVELOPMENT

This invention was made with government support under GM117058 awarded by the National Institutes of Health. The government has certain rights in the invention.

REFERENCE TO AN ELECTRONIC SEQUENCE LISTING

The content of the electronic sequence listing (339639_29-22US.xml; Size: 4,096 bytes; and Date of Creation: Aug. 15, 2023) is herein incorporated by reference in its entirety.

### BACKGROUND OF THE INVENTION

The comprehensive sequencing of proteins for the determination of their abundance and sequence variations is critical for understanding biological systems at a functional level in health and disease.¹ Mass spectrometry (MS)-based proteomics has emerged as the most powerful technique for sequencing proteins.² The ‘bottom-up’ approach, which analyzes proteins digested into peptides, enables deep proteome coverage for the identification and quantification of proteins and post-translational modifications (PTMs).³ The lop-down' approach studies intact proteoforms for comprehensive characterization of PTMs together with sequence variations.⁴⁻⁶

These complementary approaches require that proteins first be extracted and solubilized from cells. Surfactants (also known as detergents) are commonly used for cell lysis and solubilization of proteins, especially when hydrophobic species such as membrane proteins are concerned.⁷ Unfortunately, surfactants generally have a deleterious effect on MS signals and results, as well as common protein/peptide separation techniques such as reversed-phase liquid chromatography (RPLC).^{8,9}

For bottom-up proteomics, surfactants are commonly removed using filters; however, this approach can be time-consuming, labor-intensive, and result in protein loss.^{10, 11} More mild detergents, such as n-dodecyl-β-d-maltoside (DDM), have been used directly with LC-MS to increase throughput for hydrogen-deuterium exchange¹² and single-cell proteomics,¹³ as the detergent elutes away from peptides. However, this approach can cause issues with LC column fouling and method robustness.⁹ In top-down proteomics, surfactants are commonly removed by precipitation,¹⁴ which can result in sample loss and irreproducibility or fail to fully remove the surfactant.¹⁵

A promising alternative approach to filters or precipitation is MS-friendly cleavable surfactants that can extract proteins and then be degraded into innocuous byproducts before MS analysis.¹⁶ Anionic, acid-cleavable surfactants¹⁷⁻¹⁹ have been commonly employed for bottom-up proteomics and the present inventor group previously developed an anionic photocleavable surfactant amenable to both bottom-up and top-down proteomics.^{20 , 21} However, these previously developed surfactants are highly denaturing, a characteristic that is beneficial for global protein extraction and enhanced proteolytic digestion, but precludes their use for applications where non-denaturing conditions are desirable (e.g., affinity purification). Moreover, while anionic cleavable surfactants are MS-friendly after degradation and have been successfully employed for proteomics experiments, they can still suppress MS signals making them suboptimal for high-sensitivity applications compared to non-ionic detergents.²² This is especially true for the acid-cleavable surfactants, which were found to greatly suppress the signal of proteins and peptides when analyzed direly by MS.^{20,21,23}

Accordingly, there is a need to develop improved MS-compatible surfactants, particularly surfactants having desirable characteristics, including but not limited to enhanced compatibility with analytes of interest (e.g., high solubility of membrane proteins) and increased flexibility with regard to cleaving methods (e.g., easily performed chemical cleavage instead of photo-cleavage requiring exposure to specific wavelengths of light).

### SUMMARY OF THE INVENTION

The present invention provides chemically-cleavable surfactants, specifically nonionic cleavable surfactants (NCS), suitable for MS-based proteomics and analysis. As used herein, the term “redox” refers to a chemical reaction that takes place between an oxidizing substance and a reducing substance, where the oxidizing substance loses electrons in the reaction while the reducing substance gains electrons.

Conventional surfactants interfere with MS-based proteomics and peptidomics, so they must be removed before analysis, resulting in sample loss and reduced throughput. One aspect of the present invention provides nonionic cleavable surfactants (NCS) that have been designed and synthesized by inserting a cleavable disulfide bond between a hydrophilic head and hydrophobic tail, where the cleavable disulfide bond is able to release the hydrophilic head and release the hydrophobic tail when cleaved. In an embodiment, the surfactants of the present invention are able to be cleaved and degraded upon treatment with a suitable reducing agent and/or collisional and electron based fragmentation techniques. The resulting cleavage products are more easily removed prior to MS analysis and/or provide reduced MS interference. Preferably, the NCS are also nonionic and are non-denaturing in that they will not significantly denature proteins or polypeptides. Accordingly, in one application, the chemically-cleavable surfactants of the present invention are suitable for native mass spectrometry (MS) and MS analysis of proteins, protein complexes and large polypeptides. Additionally, the surfactants of the present invention are suitable for structural applications and analytical techniques other than MS, including but not limited to the preparation of samples.

General types of surfactants able to be cleaved using oxidation-reduction reactions were first published in a 2019 Langmuir paper (Xue et al., Langmuir, 2019, 35: 4319-4327) as part of a class of chemically cleavable detergents (CCDs) for use in the study of membrane proteins. In that paper, CCDs were used for selective exchange to remove membrane proteins of interest for subsequent reconstitution and analysis using electron microscopy.

However, the present disclosure is believed to be the first demonstration of NCS optimized for MS-based proteomics. The NCS described herein are able to extract and solubilize proteins, including membrane proteins, for streamlined characterization using native, top-down and bottom-up proteomics.

In an embodiment, the surfactants of the present invention are additionally designed to mimic the properties of one of the most used surfactants in structural biology, n-dodecyl-beta-d-maltoside (DDM). However, unlike DDM, the surfactants disclosed herein include a disulfide bond to enable facile cleavage and surfactant removal prior to MS analysis. Additionally, the surfactants disclosed herein are able to be used in structural applications and analytical techniques other than MS. For example, in one embodiment surfactants disclosed herein are used to prepare samples for crystallography, electron microscopy, and cryo-electron microscopy.

In one embodiment, the present invention provides a chemically-cleavable surfactant comprising: a) hydrophilic head, b) a hydrophobic tail, and c) a cleavable disulfide bond covalently linking the hydrophilic head and hydrophobic tail, where the cleavable disulfide bond is able to release the hydrophilic head, release the hydrophobic tail, and/or break apart upon exposure to a reducing agent. In an embodiment, any reducing agent may be used that is able to reduce and break disulfide bonds. Preferably, the reducing agent is selected from the group consisting of tris(2-carboxyethyl)phosphine (TCEP), dithiothreitol (DTT), beta mercaptoethanol (BME), dithiobutylamine (DTBA), [N,N′-dimethyl-N,N′-bis(mercaptoacetyl) hydrazine (DMH), glutathione (GSH), borane dimethylsulfide (BMS), cysteine-HCl, 2-mercaptoethylamine-HCl, and combinations thereof. In an embodiment, the surfactant is n-decyl-disulfide-β-D-maltoside (DSSM).

Preferably, the chemically-cleavable surfactant is stable at a pH range that is not operable for other existing surfactants, such as acid labile surfactants. In an embodiment, the chemically -cleavable surfactant is able to remain stable at a pH of 4 or lower, at a pH of 3 or lower, at a pH of or lower 2, or at a pH of 1 or lower. Conventional acid labile surfactants typically hydrolyze at a pH of approximately 2-3. In an embodiment, the chemically-cleavable surfactants of the present invention are stable at low pH (˜4). This makes surfactants of the present invention well suited for both offline and online LC/MS analysis of proteins, which commonly utilize acid in the electrospray solution and mobile phases. Moreover, many proteins need to be extracted under acidic conditions, which renders conventional rapidly acid-labile surfactants ineffective. Similarly, in an embodiment, the chemically-cleavable surfactant is stable at a pH of 7 or greater, a pH of 6 or greater, a pH of 5 or greater, a pH of 4 or greater, or a pH of 3 or greater.

In an embodiment, the chemically-cleavable surfactant has the formula:

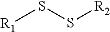

wherein, R1 is a hydrophilic moiety and R2 is a hydrophobic moiety. In an embodiment, R1 comprises one or more monosaccharides, disaccharides, polysaccharides, or combinations thereof, where at least one of the one or more saccharides are branched. In an embodiment, R1 comprises an ether group having between 1 and 30 carbon atoms, between 1 and 20 carbon atoms, between 1 and 16 carbon atoms, between 1 and 10 carbon atoms, or between 1 and 6 carbon atoms.

In an embodiment, R1 comprises an oligoglycerol, including but not limited to a triglycerol, tetraglycerol, pentaglycerol, hexaglycerol and/or decaglycerol, or R1 comprises a polyethylene glycol. In an further embodiment, R1 comprises a polyethylene glycol having the formula (≡(O—CH₂—CH₂)ₙ—OH), where n is an integer selected from 3-20, preferably selected from 3-15, or preferably selected from 4-10. As used herein, dashed lines represent the bond where a group or moiety attaches to the rest of the surfactant molecule.

In an embodiment, R1 comprises a carbohydrate having the general formula Cx(H₂O)n, or derivatives thereof, including monosaccharides and disaccharides. Suitable carbohydrates able to form R1 include, but are not limited to, derivatives of glucose (where x=6 and n=6), sucrose (where x=12 and n=11), and maltose (where x=12 and n=11) (see FIG. 21, panel i). Exemplary monosaccharides able to form R1 are illustrated in FIG. 22.

In an embodiment, R1 comprises a group having the formula:

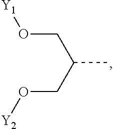

where Y1 and Y2, independently form one another, are each a monosaccharide, disaccharide, or polysaccharide (see FIG. 21, panel ii). The dashed line represents the bond where the group attaches to the rest of the surfactant molecule. Optionally, Y1 and Y2 each have a maximum of four saccharide groups. Preferably, Y1 and Y2 combined have a maximum of four saccharide groups.

In an embodiment, R1 comprises an oligoglycerol having the formula:

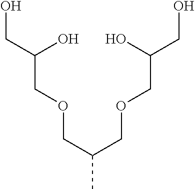

(see FIG. 21, panel iii). The dashed line represents the bond where the oligoglycerol attaches to the rest of the surfactant molecule.

In an embodiment, R1 comprises a polyethylene glycol having the formula:

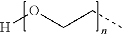

(see FIG. 21, panel iv). Preferably, n is an integer selected from 3-20, selected from 3-15, or selected from 4-10. The dashed line represents the bond where the polyethylene glycol attaches to the adjacent group or the rest of the surfactant molecule.

In an embodiment, R2 is a substituted or unsubstituted alkyl, alkenyl, alkynyl, or aryl group having between 1 and 40 carbon atoms, between 1 and 30 carbon atoms, between 1 and 20 carbon atoms, between 1 and 16 carbon atoms, between 1 and 10 carbon atoms, or between 1 and 6 carbon atoms. In an embodiment, R2 is a substituted or unsubstituted alkyl, alkenyl, alkynyl, or aryl group between 2 and 40 carbon atoms, between 2 and 30 carbon atoms, between 2 and 20 carbon atoms, between 2 and 16 carbon atoms, between 2 and 10 carbon atoms, between 2 and 6, between 6 and 20 carbon atoms, between 6 and 15 carbon atoms, or between 6 and 10 carbon atoms. Optionally, the alkyl, alkenyl, alkynyl, or aryl groups are branched or unbranched.

In an embodiment, R2 comprises an alkyl group having the formula:

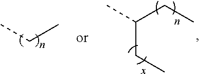

where n and x, independently from one another, are integers selected from 1-30, preferably selected from 1-20, or preferably selected from 1-15. The dashed line represents the bond where the alkyl group attaches to the rest of the surfactant molecule.

In an embodiment, R2 comprises an aryl group having the formula:

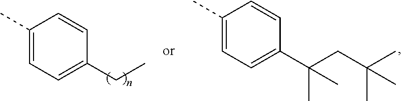

where n is an integer selected from 1-30, preferably selected from 1-20, preferably selected from 1-15. The dashed line represents the bond where the aryl group attaches to the rest of the surfactant molecule.

Optionally, the chemically-cleavable surfactant comprises a linker group between R1 and the disulfide bond, where the linker is an ether, alkyl, alkenyl, or alkynyl group having between 1 and 12 carbon atoms. Preferably, the linker is an alkyl group having between 1 and 6 carbon atoms.

In an embodiment, the chemically-cleavable surfactant has the formula:

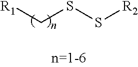

wherein, R1 is a hydrophilic group as defined above, R2 is a hydrophobic group as defined above, and n is an integer selected from 1 to 6. Preferably, R1 comprises one or more branched or unbranched monosaccharides, disaccharides, or polysaccharides, an oligoglycerol, a polyethylene glycol, or combinations thereof. Preferably, R2 is a substituted or unsubstituted alkyl, alkenyl, alkynyl, or aryl group having between 2 and 30 carbon atoms.

In an embodiment, the chemically-cleavable surfactant has the formula:

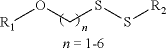

wherein, R1 is a hydrophilic group as defined above, R2 is a hydrophobic group as defined above, and n is an integer selected from 1 to 6. Preferably, R1 comprises one or more branched or unbranched monosaccharides, disaccharides, or polysaccharides, an oligoglycerol, a polyethylene glycol, or combinations thereof. Preferably, R2 is a substituted or unsubstituted alkyl, alkenyl, alkynyl, or aryl group having between 2 and 30 carbon atoms.

In an embodiment, the chemically-cleavable surfactant has the formula:

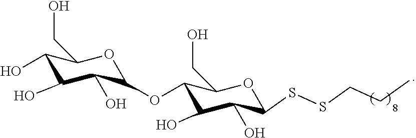

In an embodiment, the present invention provides a method of solubilizing a compound comprising the steps of: a) mixing the compound with a chemically-cleavable surfactant in a solution until the compound is dissolved in the solution; and b) exposing the solution containing the chemically-cleavable surfactant and dissolved compound to a reducing agent, thereby cleaving the chemically-cleavable surfactant, wherein the chemically-cleavable surfactant comprises: i) hydrophilic head, ii) a hydrophobic tail, and iii) a cleavable disulfide bond covalently linking the hydrophilic head and hydrophobic tail, where the cleavable disulfide bond is able to release the hydrophilic head, release the hydrophobic tail, and/or break apart upon exposure to a reducing agent. In an embodiment, any reducing agent may be used that is able to reduce and break the disulfide bonds. Preferably, the reducing agent is selected from the group consisting of tris(2-carboxyethyl)phosphine (TCEP), dithiothreitol (DTT), beta mercaptoethanol (BME), dithiobutylamine (DTBA), [N,N′-dimethyl-N,N′-bis(mercaptoacetyl) hydrazine (DMH), and combinations thereof.

The solution comprises an organic solvent, aqueous solvent, or combinations thereof. Optionally, the solution also comprises 10% or less of an acid. The solution also comprises 5% or less of the chemically-cleavable surfactant, preferably 1% or less of the photo-cleavable surfactant, preferably 0.5% or less of the chemically-cleavable surfactant. In an embodiment, the solution comprises 70% or more of an organic solution, 5% or less of an acid, and 0.1% or less of the chemically-cleavable surfactant. Preferred organic solvents include, but are not limited to, acetonitrile, methanol, and isopropanol (IPA).

Preferably, the compound is a protein, protein complex, or polypeptide. In an embodiment, the compound is a membrane protein, including but not limited to receptor proteins (e.g., beta-adrenergic receptors) or ion channel proteins. In an embodiment, the compound is an extra-cellular matrix protein (ECM).

Optionally, the method further comprises performing mass spectrometry (MS) analysis on a portion of the solution containing the compound and cleaved chemically-cleavable surfactant, such as part of an online liquid chromatography-mass spectrometry (LC/MS) experiment. In an embodiment, after dissolving the compound in a solution containing the chemically-cleavable surfactant, a chromatography step is performed on the solution in order to purify or separate the components in the solution. The portion or fraction of the solution which contains the dissolved compound is exposed to the reducing agent before injecting or spraying the dissolved compound into the mass spectrometer. Optionally, the cleaving step occurs after chromatography separation of proteins before spraying into the mass spectrometer, as the experiments happen in a real time.

The chemically-cleavable surfactants of the present invention are also amendable for use in automated MS systems, where the cleavage of the surfactant is able to be performed within the MS device itself and optionally without the use of a reducing agent. For example, in an embodiment, collision and electron based fragmentation techniques including but not limited to collision-induced dissociation (CAD), electron transfer dissociation (ETD), electron capture dissociation (ECD) used in common MS analysis are used to cleave the surfactant and dissociate the surfactant and/or its cleavage products from the compound.

Accordingly, in an embodiment, the chemically-cleavable surfactants of the present invention are provided in a method for analyzing a compound comprising the steps of:

a) mixing the compound with a chemically-cleavable surfactant in a solution until the compound is dissolved in the solution, wherein the chemically-cleavable surfactant comprises: i) a hydrophilic head, ii) a hydrophobic tail, and iii) a cleavable disulfide bond covalently linking the hydrophilic head and hydrophobic tail, where the cleavable disulfide bond is able to release the hydrophilic head, release the hydrophobic tail, and/or break apart upon exposure to collisional energy during mass spectrometry;

b) exposing the solution containing the chemically-cleavable surfactant and dissolved compound to collisional energy or electron based fragmentation within a mass spectrometer device, thereby decomposing the chemically-cleavable surfactant; and

c) performing mass spectrometry (MS) analysis on a portion of the exposed solution containing the compound. In an embodiment, exposing the solution containing the chemically-cleavable surfactant and dissolved compound to collisional energy comprises performing collision-induced dissociation (CID). Preferably, the solution containing the chemically-cleavable surfactant and dissolved compound is exposed to collisional energy between 2-200 eV, between 20-200 eV, between 60-200 eV, between 100-200 eV, between 150-200 eV, between 60-150 eV, between 20-60 eV, between 20-50 eV, between 20-30 eV, or between 2-10 eV.

Preferably, the compound comprises one or more polypeptides and/or intact soluble protein complexes. Preferably, the compound as a molecular weight of 100 daltons or more, 200 daltons or more, 500 daltons or more, or 1,000 daltons or more.

In an embodiment, the chemically-cleavable surfactant of the above methods has the formula:

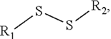

wherein, R1 is a hydrophilic moiety and R2 is a hydrophobic moiety. In an embodiment, R1 comprises between 1 and 30 carbon atoms, between 1 and 20 carbon atoms, between 1 and 16 carbon atoms, between 1 and 10 carbon atoms, or between 1 and 6 carbon atoms. Preferably, R1 comprises at least two carbon atoms.

Optionally, R1 comprises one or more branched or unbranched monosaccharides, disaccharides, polysaccharides, or combinations thereof. Optionally, R1 comprises a group having the formula:

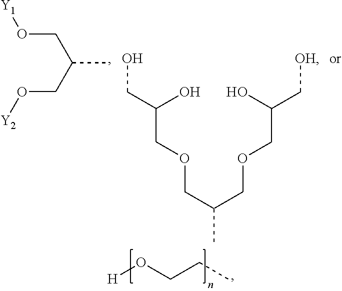

where n is an integer selected from 3-20, selected from 3-15, or selected from 4-10, and where Y1 and Y2, independently form one another, are each a monosaccharide, disaccharide, or polysaccharide. The dashed line represents the bond where the group attaches to the adjacent group or the rest of the surfactant molecule. Preferably, R1 comprises one or more monosaccharides, disaccharides, polysaccharides, or combinations thereof.

In an embodiment, R2 is a substituted or unsubstituted alkyl, alkenyl, alkynyl, or aryl group having between 1 and 40 carbon atoms, between 1 and 30 carbon atoms, between 1 and 20 carbon atoms, between 1 and 16 carbon atoms, between 1 and 10 carbon atoms, between 1 and 6 carbon atoms, between 6 and 20 carbon atoms, between 6 and 15 carbon atoms, or between 6 and 10 carbon atoms. Preferably, R1 comprises at least two carbon atoms. Preferably, R2 has the formula:

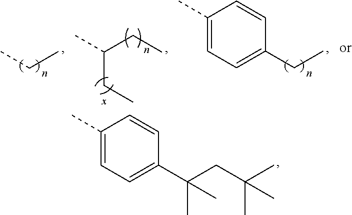

where n is an integer selected from 1-30, preferably selected from 1-20, preferably selected from 1-15. The dashed line represents the bond where the alkyl group or aryl group attaches to the rest of the surfactant molecule.

In an embodiment, wherein the chemically-cleavable surfactant of the above

methods has the formula:

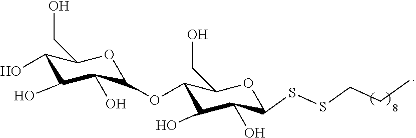

Accordingly, the NCS reagents and methods of the present invention provide valuable tools for MS-based proteomics, with applications ranging from in-depth proteoform characterization to proteome-wide studies. The NCS reagents disclosed herein are useful as surfactants for improved single cell analysis and for use in LC-MS of standard intact proteins, top-down proteomics of membrane proteins and cell lysates, and single-pot bottom-up proteomics.

Additionally, the surfactants and methods described herein can be utilized to improve solubility of compounds in a wide range of structural biological and chemical applications, including applications that typically involve denaturing surfactants. For example, to study integral membrane proteins, the proteins must be extracted from the membrane, a step that is typically achieved by the application of detergents (see Stetsenko et al., Crystals, 2017, 7(7):197). Once removed, the extracted proteins may be used to prepare samples for analytical methods, including but not limited to x-ray crystallography, electron microscopy, and cryo-electron microscopy. In an embodiment, the surfactants disclosed herein are used as a replacement for conventional surfactants (such as DDM) in the preparation of samples for x-ray crystallography and electron microscopy, particularly cryo-electron microscopy. Similarly, hydrogen deuterium exchange mass spectrometry (MS) has become a key technique for monitoring structural and dynamic aspects of proteins dissolved in solution (see Konermann et al., Chem. Soc. Rev., 2011, 40(3): 1224). In another embodiment, the surfactants disclosed herein are used as a replacement for conventional surfactants (such as DDM) currently used in hydrogen deuterium exchange-MS.

### BRIEF DESCRIPTION OF THE DRAWINGS

FIG. 1. (Panel A) Comparison of the chemical structures of n-Dodecyl-β-D-maltoside (DDM) and n-Decyl-disulfide-β-D-maltoside (DSSM). (Panel B) Overview of using a DSSM for proteomics.

FIG. 2. Compatibility of DSSM with ESI-MS of intact proteins. MS spectra of carbonic anhydrase (left), carbonic anhydrase with DDM (2× CMC) (middle), and carbonic anhydrase in DSSM after degradation with TCEP (right).

FIG. 3. NMR of the final product. ¹H NMR (400 MHz, DMSO-d₆) δ 5.63 (d, J=3.2 Hz, 1H), 5.46 (d, J=6.1 Hz, 1H), 5.33 (d, J=6.1 Hz, 1H), 5.02 (d, J=3.8 Hz, 1H), 4.90 (t, J=5.2 Hz, 2H), 4.52 (t, J=5.4 Hz, 1H), 4.45 (t, J=5.6 Hz, 1H), 4.32 (d, J=9.5 Hz, 1H), 3.70 (dd, J=11.4, 5.3 Hz, 1H), 3.62 (dd, J=9.7, 5.3 Hz, 1H), 3.54 (dt, J=11.4, 5.4 Hz, 1H), 3.50-3.42 (m, 3H), 3.41-3.35 (m, 1H), 3.31-3.26 (m, 3H), 3.23 (ddt, J=9.7, 6.1, 2.8 Hz, 1H), 3.06 (td, J=9.1, 5.6 Hz, 1H), 2.80 (td, J=7.0, 1.9 Hz, 2H), 1.61 (p, J=7.1 Hz, 2H), 1.36-1.19 (m, 14H), 0.89-0.82 (m, 3H).

FIG. 4. ESI-MS for DSSM (M=C₂₂H₄₂O₁₀S₂). Experimental=[M+NH₄]⁺=548.259 Da, Calculated=548.256 Da, Error=0.003 Da. Minor peaks [2M+H]⁺. 1061.457, [2M+NH₄]⁺=1078.457.

FIG. 5. ESI-MS of carbonic anhydrase with and without DDM.

FIG. 6. Time-course of DSSM (2× CMC) degradation using MS signal of carbonic anhydrase as a benchmark. DSSM was degraded with 5 eq of TCEP. The signal of carbonic anhydrase was monitored to determine the time necessary to achieve a quality MS signal.

FIG. 7. Picture of DSSM (10× CMC in water) before and after degradation with TCEP. The sample was centrifuged, and no precipitate was observed.

FIG. 8. ESI-MS analysis of carbonic anhydrase in DSSM at 2× and 20× CMC.

FIG. 9. (Panel A) Schematic representation of surfactant dissociation using collisional activation. (Panels B-C) Native MS of carbonic anhydrase monomers and the tetramer forming alcohol dehydrogenase complex with and without DSSM (2× CMC or 5× CMC) under different collisional activation conditions.

FIG. 10. ESI-MS analysis of carbonic anhydrase with and without DDM or DSSM (2× CMC).

FIG. 11. ESI-MS analysis of alcohol dehydrogenase with and without DSSM (2× CMC).

FIG. 12. Comparison of DSSM and DDM compatibility with LC-MS analysis of intact protein mixture. (Panel A) Schematic representation of LC-MS analysis of intact protein in DSSM, (Panel B) Chromatogram of ribonuclease A (RiA), myoglobin (Myo), and carbonic anhydrase (CA). Three replicates are overlapped for each condition. (Panels C-D) Mass spectrum of intact proteins in DSSM (after degradation) and DDM.

FIG. 13. Base peak chromatogram of a mixture of standard proteins in DSSM after degradation demonstrating the elution of the head group in the void volume before protein elution.

FIG. 14. Top-down proteomics of DSSM solubilized membrane proteins. Intact mass spectra and fragmentation map for analysis of KcsA (Panels A-B) and bacteriorhodopsin (Panels C-D). Proteins were solubilized in DSSM and analyzed by LC-MS/MS after surfactant degradation. The polypeptide illustrated in panel B has the amino acid sequence of SEQ ID NO:1, and the polypeptide illustrated in panel D has the amino acid sequence of SEQ ID NO:2.

FIG. 15. Representative fragment spectra for LC-MS/MS analysis of KcsA with a theoretical fit.

FIG. 16. Representative fragment spectra for LC-MS/MS analysis of bacteriorhodopsin with a theoretical fit.

FIG. 17. Native MS of bacteriorhodopsin in C8E4 or DSSM (labelled as “RCS” in the figure) detergent using different voltage conditions. Surfactant structures are shown on the right.

FIG. 18. Top-down proteomics of endogenous proteins extracted from cell lysate using DSSM. (Panels A-D) Representative proteins were confidently identified from HEK whole cell or crude membrane lysate (Panels A, B, and D from whole cell lysate and Panel C from crude membrane lysate).

FIG. 19. (Panel A) Workflow for single-pot DSSM-aided (labelled as “RCS” in the figure) bottom-up proteomics. (Panel B) Protein groups were identified across three extraction replicates using 2× and 20× CMC DSSM.

FIG. 20. Evaluation of the quantitative reproducibility of DSSM-aided bottom-up proteomics. Protein intensity correlation plots for 2× CMC extraction replicates (Panel A), 20× CMC extraction replicates (Panel B), and 2× compared to 20× (Panel C).

FIG. 21. Exemplary saccharide, oligoglycerol and polyethylene glycol structures for hydrophilic head groups.

FIG. 22. Exemplary monosaccharides able to form hydrophilic head groups of the present invention.

### DETAILED DESCRIPTION OF THE INVENTION

Overview

Surfactants are valuable tools for enabling the characterization of proteins by facilitating their extraction and purification. However, the presence of surfactants, even mild surfactants like n-dodecyl-β-D-maltoside (DDM), often has a deleterious effect on top-down proteomics for protein sequencing to identify post-translational modifications (PTMs) and sequence variations.^{4,8,9} Surfactant-related signal suppression is generally caused by the higher ionization efficiency and signal-to-noise ratio of the low molecular weight species. Moreover, the presence of surfactant can negatively impact common front-end protein separation techniques such as reversed-phase liquid chromatography (RPLC), which could cause potential problems in reproducibility and robustness.^{4,9} Accordingly, there is a need to develop improved MS-compatible surfactants.

Nonionic cleavable surfactants (NCS) are versatile tools for MS-based proteomics, with applications ranging from in-depth proteoform characterization to proteome-wide studies severing as a general replacement for DDM. Given the role of surfactants for protein purification and proteomics, the NCS disclosed herein are a valuable tool for basic and translation research.

The examples below present the first application of NCS for MS-based proteomics, and demonstrates the ability of NCS to extract and solubilize proteins, including membrane proteins, for streamlined characterization using native, top-down, and bottom-up proteomics. Similar surfactants used for biochemistry and biophysical procedures interfere with MS analysis of proteins and peptides and therefore must be removed before analysis, which can result in sample loss and lower throughput.

### Example 1

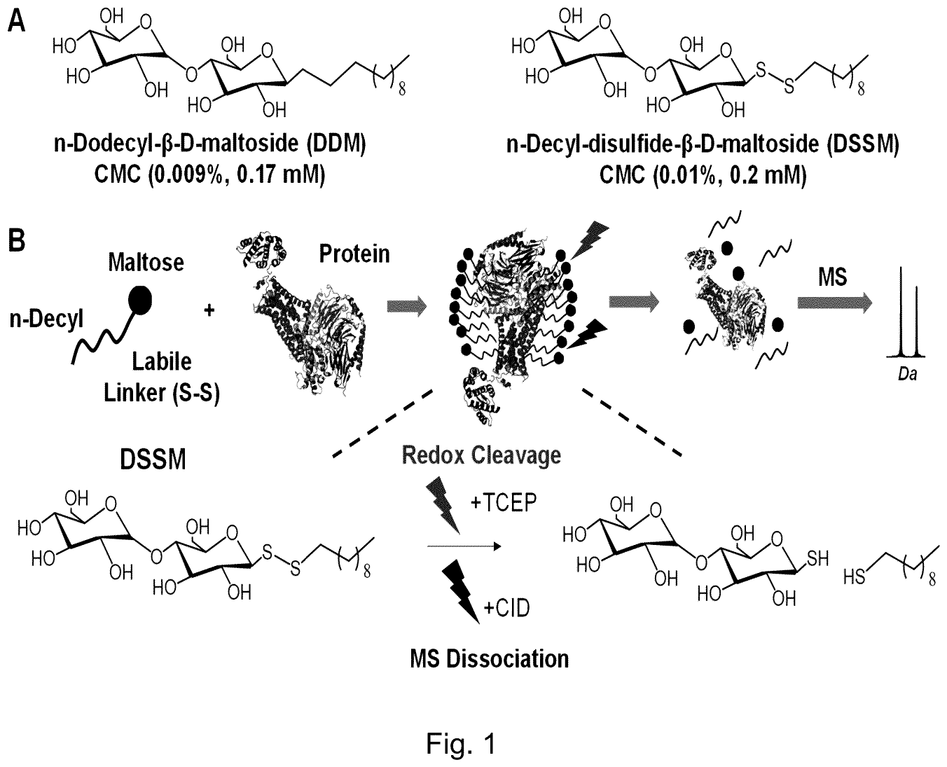

One approach to overcome the incompatibility of the surfactants for downstream proteomic analysis is to insert a cleavable bond (e.g., an acid-labile¹⁷⁻¹⁹ or light-labile^{20,21} bond) that allows for controlled degradation of the molecule into innocuous byproducts before MS analysis. Unfortunately, cleavable surfactants commonly used for proteomics contain denaturing, anionic head groups, such as sulfate, that preclude their use for applications where non-denaturing conditions are desirable.^{12,31} Thus, there is an urgent need for cleavable surfactants that can aid in traditional biochemical preparation methods under non-denaturing conditions yet are still amenable for downstream proteomic applications. This example demonstrates for the first time the use of n-decyl-disulfide-β-D-maltoside (DSSM), a nonionic, cleavable surfactant, for use with top-down proteomics and MS analysis (FIG. 1).

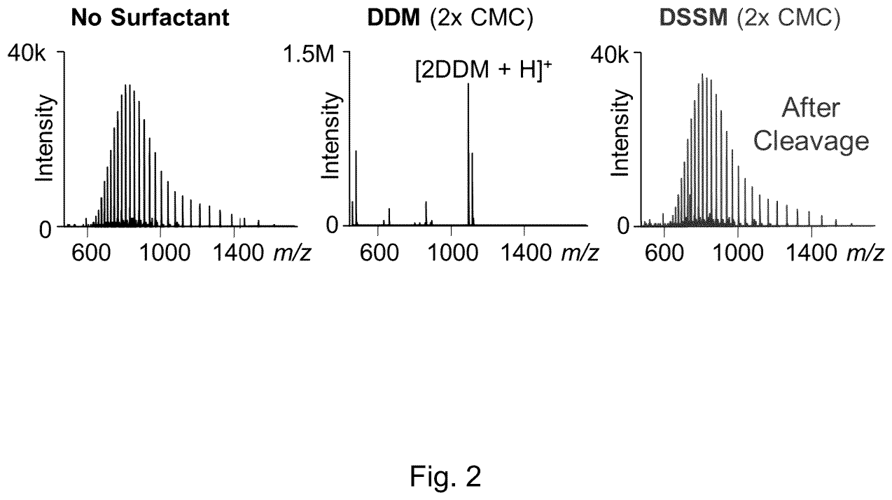

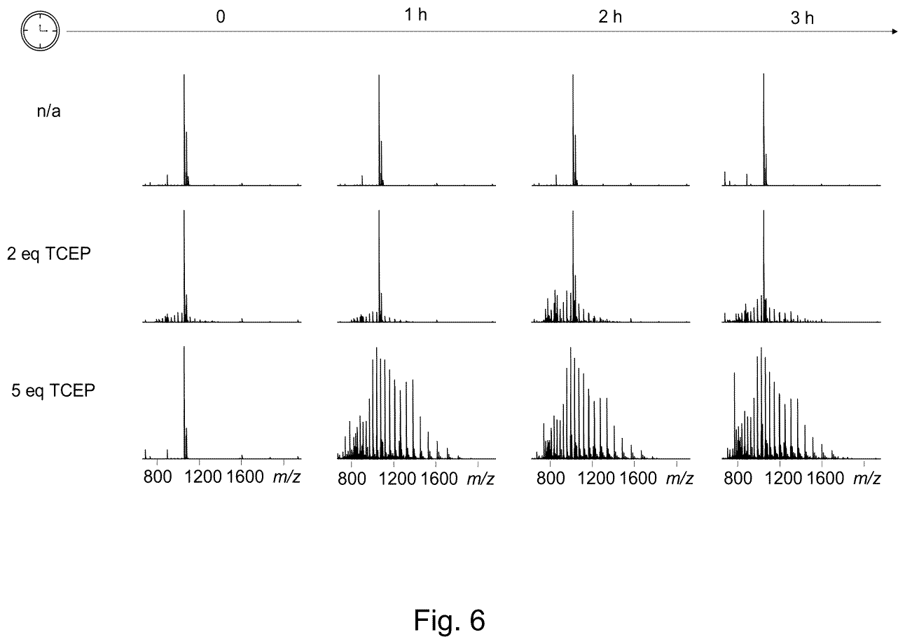

DSSM was originally developed to mimic the properties of DDM while providing a platform for high-throughput detergent exchange for biophysical assays.²⁴ The nonionic maltose head group resembles DDM, but the addition of the disulfide bond between the sugar and the hydrophobic decyl tail imparts cleavable properties. After synthesis and characterization of DSSM (FIGS. 2 and 6), it was evaluated for compatibility with electrospray-ionization (ESI)-MS analysis of intact proteins.

Methods

Chemicals and Materials. All chemicals and reagents were purchased from Millipore Sigma Inc. (St. Louis, MO, USA) unless noted otherwise. Bicinchoninic acid (BCA) reagent, Dulbecco's modified Eagle's medium (DMEM), fetal bovine serum (FBS), halt protease/phosphatase inhibitor cocktail, penicillin-streptomycin, phosphate-buffered saline (PBS), tris(2-carboxyethyl)phosphine (TCEP), were obtained from Thermo Fisher Scientific (Waltham, MA, USA). HPLC-grade water, isopropanol, formic acid, and acetonitrile (ACN) were obtained from Fisher Scientific (Fair Lawn, NJ, USA). Human embryonic kidney (HEK) 293T cells were purchased from American Type Culture Collection (ATTC, Manassas, VA, USA). n-Dodecyl-β-D-maltoside (DDM) and n-Decyl-disulfide-β-D-maltoside (DSSM). solutions were to 1% (w/v) in 150 mM LC-MS grade ammonium acetate pH 7.

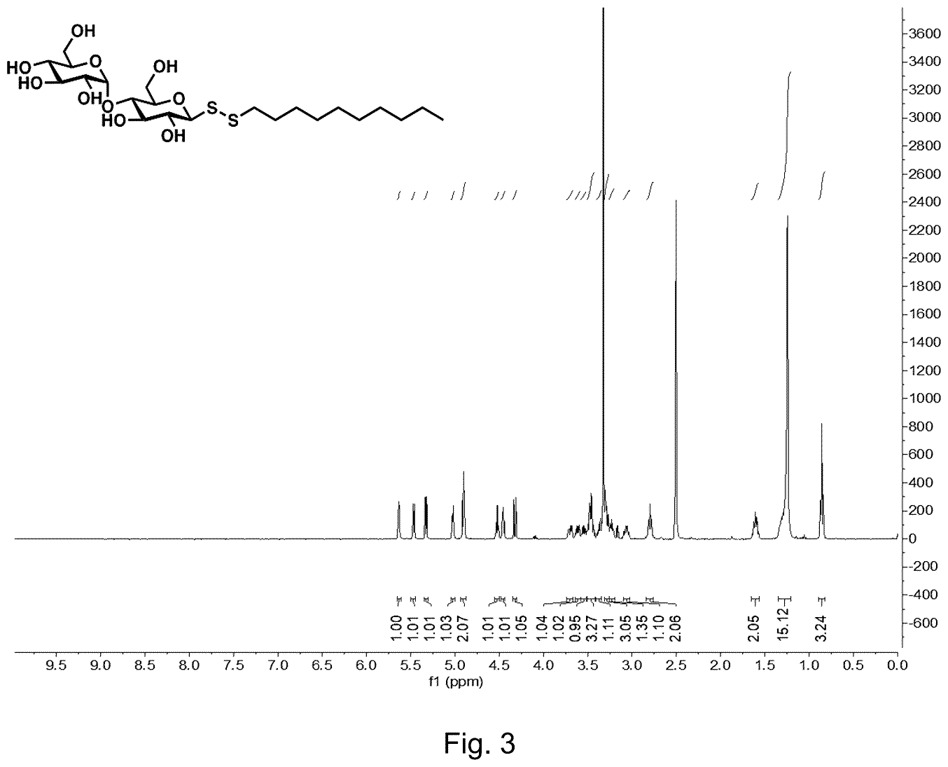

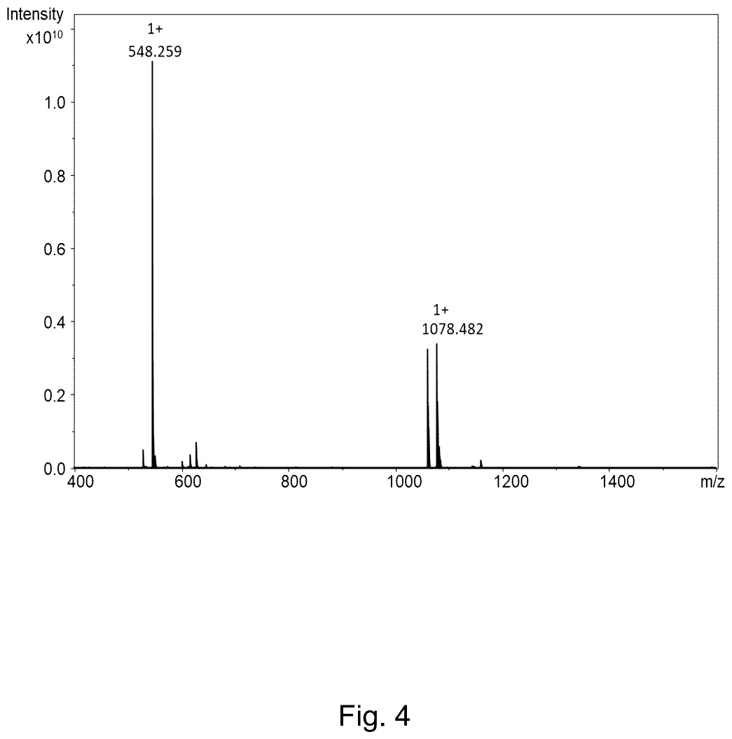

DSSM synthesis and characterization. The DSSM synthetic method was adapted from Xue et al. 24 and the final product was characterized using NMR (FIG. 3) and electrospray ionization (ESI)-MS (FIG. 4). The detailed synthesis procedure is given in the supporting information in Example 2 below.

ESI-MS of standard intact protein. Carbonic Anhydrase from bovine erythrocytes was dissolved in 50:49:1 water:methanol:formic acid supplemented with DSSM (0.4 mM) or DDM (0.5 mM) at a final concentration of 0.05 mg/mL. 2 mM TCEP was added to the samples and incubated at 4° C. for 2 h. Samples were infused with a nano-ESI sprayer (TriVersa NanoMate; Advion Bioscience) using a voltage of 1.4-1.6 kV versus the inlet and 0.3-0.5 psi drying gas into a Bruker solariX 12T FTICR-MS (Bruker Daltonics). Mass spectra were acquired from 200-3,000 m/z for 100 scans.

Carbonic anhydrase from bovine erythrocytes and alcohol dehydrogenase from yeast were dissolved in 150 mM ammonium acetate. Samples were desalted using an Amicon molecular weight cut-off filter with 150 mM ammonium acetate. DSSM was then added to 0.4 mM-1 mM with a protein concentration of 10 μM. Samples were infused with a nano-ESI sprayer (TriVersa NanoMate; Advion Bioscience) using a voltage of 1.4-1.6 kV versus the inlet and 0.3-1.5 psi drying gas into a Bruker solariX 12T FTICR-MS (Bruker Daltonics). Data were collected with 5×10⁵ data points at 200-8000 m/z.

LC-MS of standard intact proteins. Ribonuclease A (RiA), Myoglobin (Myo), Carbonic Anhydrase (CA) from bovine erythrocytes made to 0.05 mg/mL in 25 mM ammonium bicarbonate (ABC) with or without surfactant (2× and 20× CMC). 10 mM TCEP was used to degrade the DSSM at 4° C. for 2 h. 1 μL was injected onto a home-packed PLRP capillary column (250×0.250 mm, 5 μm, 1000 Å) heated to 5° C. The separation was performed with a mobile phase of water+0.2% formic acid (A) and acetonitrile:isopropanol (1:1)+0.2% formic acid (B) using a flow rate of 5 μL/min on a Waters nanoAcquity HPLC (M-Class). Separation was performed using a 30 min gradient of 5-75% B. Proteins eluting were infused into a maXis II ETD Q-TOF or Impact II Q-TOF (Bruker Daltonics) via ESI with a capillary voltage of 4500 V and an endplate offset of 500 V. MS1 scans were collected at 1 Hz.

Top-down proteomics of membrane proteins. The detailed procedure for purification of KcsA can be found in Example 2 below. The protein was precipitated using the chloroform:methanol:water approach 25 and solubilized in DSSM (2× CMC). An aliquot of KcsA was diluted with 1 volume of water or IPA and the surfactant degraded with TCEP. 5 μL of the sample was separated using a home-packed PLRP capillary column heated to 50° C. with a 40 min gradient of 15-95% B. The eluted proteins were infused into a maXis II ETD Q-TOF or Impact II Q-TOF (Bruker Daltonics) via ESI with a capillary voltage of 4500 V and an endplate offset of 500 V. MS1 scans were collected at 0.5 Hz. The top two most intense ions were selected for collision-induced dissociation (CID) at 1-2 Hz using collisional energies ranging from (20-50 eV) depending on m/z and charge. Ions were excluded after four scans.

Bacteriorhodopsin was buffer exchanged ten times in DSSM (2× CMC) using a 100 kDa molecular weight cutoff filter (Amicon). An aliquot was diluted with 1 volume of water or isopropanol (IPA) and the surfactant degraded with TCEP. LC-MS/MS was performed using the same conditions at KcsA. Fragment ions were assigned using a tolerance of 25 ppm and manually validated using MASH Explorer (V 1.0.0.30286).²⁶

Top-down proteomics of cell lysate. 293T cells were grown on 10-cm plates with 10% FBS and 1× Penicillin-Streptomycin at 37° C. with 5% CO₂. Plates (˜95% confluent) were washed and harvested with PBS and pelleted at 500 g. The cells were lysed with DSSM (50× CMC) supplemented with lx protease/phosphatase inhibitor cocktail and 10 mM methionine. The cells were sonicated in a water bath at 4° C. for 1 h and insoluble material was removed by centrifugation (20,000 g, 10 min, 4° C.). The protein concentration was determined using a BCA assay with albumin as a standard.

Proteins were also extracted from crude membranes using a protocol adapted from Abcam (https://www.abcam.com/protocols/subcellular-fractionation-protocol). Briefly, cells from two 10-cm plates were harvested and lysed in 25 mM ABC supplemented with 1× protease/phosphatase inhibitor cocktail and 10 mM methionine passaging through a 27 G syringe ten times. The sample was centrifuged (20,000 g, 10 min, 4° C.) and the supernatant was discarded. The pellet was treated with 0.5% DSSM supplemented with 1× protease/phosphatase inhibitor cocktail and 10 mM methionine and sonicated in a water bath at 4° C. for 1 h. The insoluble material was removed by centrifugation (20,000 g, 10 min, 4° C.) and the protein concentration was determined using a BCA assay with albumin as a standard.

An aliquot of the protein extract was diluted to 1 μg/μL in water and the surfactant degraded with 5 eq of TCEP overnight at 4° C. The sample was centrifuged (20,000 g, 10 min, 4° C.) to remove insoluble material. Alternatively, the solution was concentrated to dryness using a vacuum concentrator and stored at −80° C. Just before analysis, the dried pellet was dissolved in 80% formic acid supplemented with 10 mM methionine and analyzed by LC-MS/MS.

10 μL of the sample was injected onto a home-packed PLRP capillary (250×0.250 mm, 5 μm, 1000 Å) heated to 50° C. and separated using a gradient of 15-65% over 50 min. The MS conditions were the same as stated above. For MS/MS, the top three most intense ions were selected for collision-induced dissociation (CID) at 2-4 Hz using collisional energies ranging from (20-50 eV) depending on the selected m/z and charge. Ions were excluded after four scans.

Data were analyzed as described previously in DataAnalysis 4.3 (Bruker Daltonics). Briefly, an MSalign file was created using the Sophisticated Numerical Annotation Procedure (SNAP) peak-picking algorithm with the following parameters: quality factor (0.4); signal-to-noise ratio (S/N) (3); intensity threshold (500); retention window (1.5 min). The generated file contained the following information: precursor mass, precursor charge, precursor mass followed by the fragment masses, intensities, and charges. Searches were performed in TopPIC (V1.1.2) 27 against the reviewed Homo Sapiens UniProt 28 sequences (www.uniprot.org, 2021-10-21, 20371 entries) using the default parameters.

Single-pot bottom-up. A 10 cm plate of cells was washed and harvested in PBS. The cells were suspended into 1 mL of PBS and aliquoted into 10 tubes. The cells were pelleted by centrifugation (500 g, 10 min) and the proteins were extracted with NCS (2× and 20× CMC in 25 mM ABC) at 50° C. for 1 h. Three extraction replicates were performed for each condition. The proteins were reduced with 20 mM TCEP, alkylated with 40 mM 2-chloroacetamide, and digested with 250 ng trypsin. After 1 h an additional 250 ng of trypsin was added and the samples were digested for 2 h. The digest was quenched with formic acid and samples were centrifuged to remove cell debris.

Approximately 200 ng of peptide were separated using an Ion Optics column (25 cm ×75 μm, C18, 1.6 μm) heated to 55° C. with the following gradient: 0-60 min 2-17% B, 60-90 min 17-25% B, 90-100 min 37% B, 100-110 min 37-85% B, and 110-120 min 85% B using a flow rate of 400 nL/min and mobile phase B consisting of 99.8% ACN and 0.2% formic acid. Eluting peptides were directly ionized via electrospray ionization (CaptiveSpray) using a capillary voltage of 1500V, dry gas of 3.0 l/min, and dry temp of 180° C. Ions measured from 100-1700 m/z using a timsTOF Pro Q-TOF (Bruker Daltonics) operating in PASEF mode.²⁹ The ion mobility range (1/k0) was set to 0.60-1.60 Vs/cm². For tandem MS, the following parameters were used: total cycle time (1.16 s); number of PASEF MS/MS scans (10); target intensity (20000); intensity threshold (2500); charge range (0-8); isolation width (2 m/z for m/z<700 and 3 m/z for m/z>700); collisional energy (20-59 eV). Precursor ions were excluded within a 0.4 min retention window unless the intensity reached 4-fold of the previous ion selection.

Data were processed using MSFragger V17.1 software.³⁰ For database searches, the reviewed Homo Sapiens UniProt sequences (www.uniprot.org, 2021-10-21, 20371 entries) were used using a 1% false discovery rate. All searches were performed with carbamidomethyl (C) set as a fixed modification and oxidation (M) and protein N-terminal acetylation set as variable modifications. Match between runs was enabled. Otherwise, the MSFragger parameters were not changed from their default values.

Results & Discussion

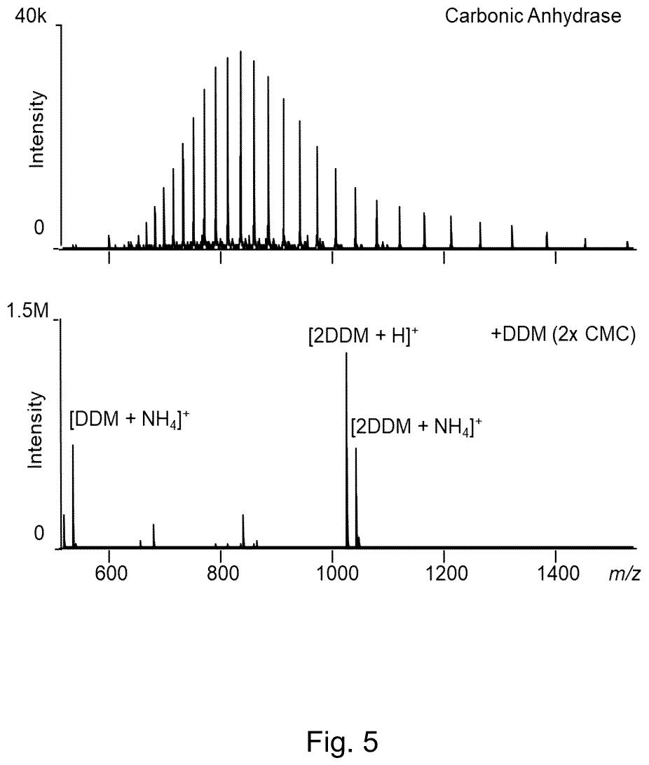

Evaluating the Compatibility of NCS for ESI-MS of Intact Protein Analysis. Nonionic surfactants are often used as general reagents for cell lysis as well as useful tools for solubilizing, stabilizing, and purifying proteins for downstream characterization.⁷ However, the presence of surfactants, even more, mild nonionic surfactants, in the sample matrix has a deleterious effect on ESI-MS of proteins.⁸ Surfactant-related signal suppression is generally caused by the higher ionization efficiency and signal-to-noise ratio of the low molecular weight species. Even at the relatively low concentration of 2× the CMC (˜0.02%), DDM is the dominant species suppressing intact carbonic anhydrase MS signal (FIG. 5). Thus, there is a significant need for cleavable surfactants that can aid in traditional biochemical preparation methods yet still be amenable for downstream applications involving mass spectrometry.

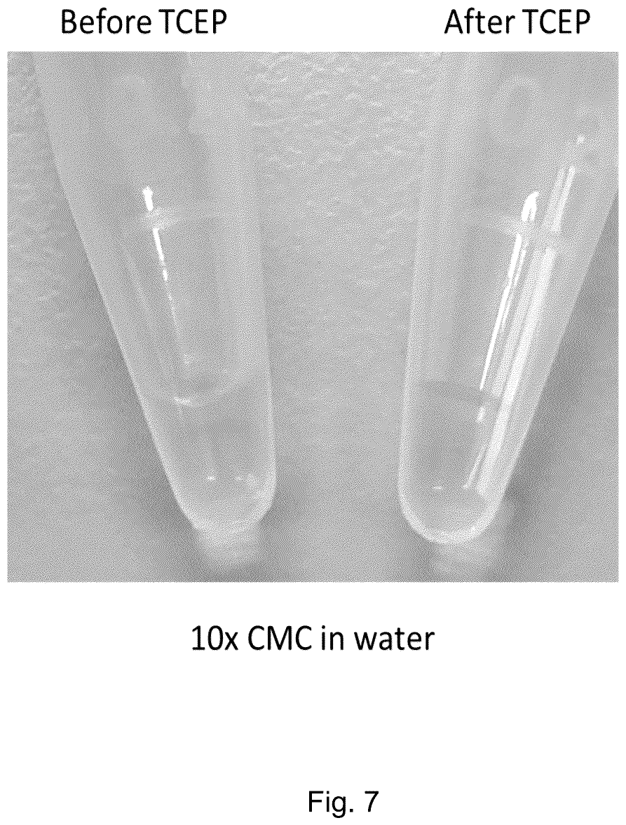

The compatibility of DSSM with direct ESI-MS analysis was evaluated using carbonic anhydrase (29.1 kDa) in denaturing conditions. The surfactant was degraded with 5 equivalence of TCEP (tris(2-carboxyethyl)phosphine) at 4° C. for 2 h (FIG. 6). In-soluble degradation products, which commonly pose an issue for acid-cleavable surfactants like RapiGest,¹⁷ were not observed after DSSM degradation and centrifugation (FIG. 7). No difference was observed in signal between the control sample and that with DSSM after degradation (FIG. 2).

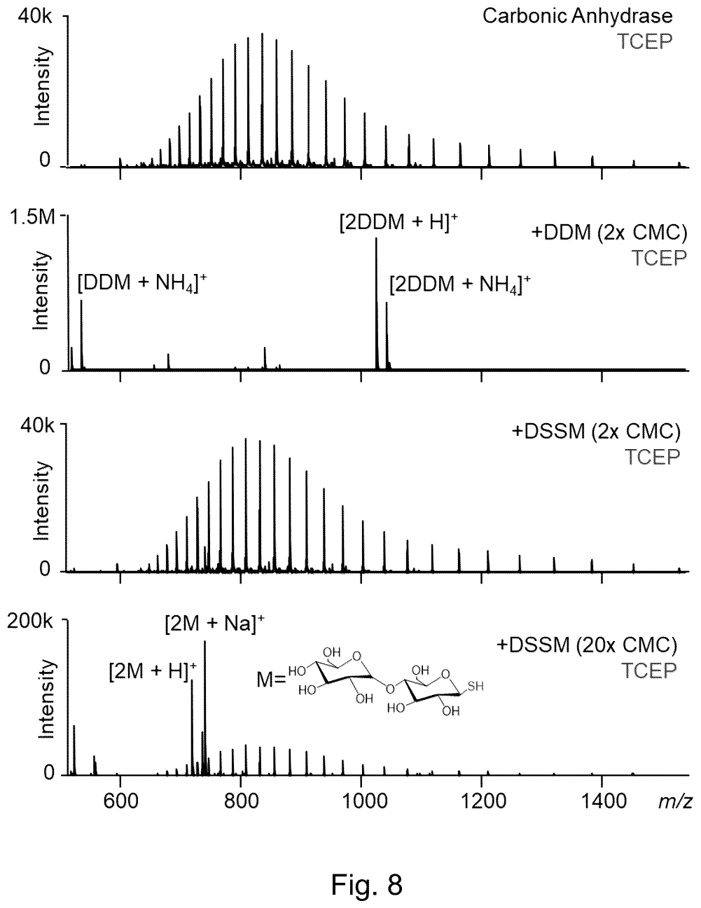

In contrast, even at the relatively low concentration of 2× CMC (0.02%), DDM is the dominant species suppressing intact-mass analysis of carbonic anhydrase (FIG. 2, middle panel). When a large excess of DSSM was used (20× CMC), a species corresponding to the maltose head group was observed as the dominant peak (FIG. 8). No deleterious effects were observed from the inclusion of TCEP.

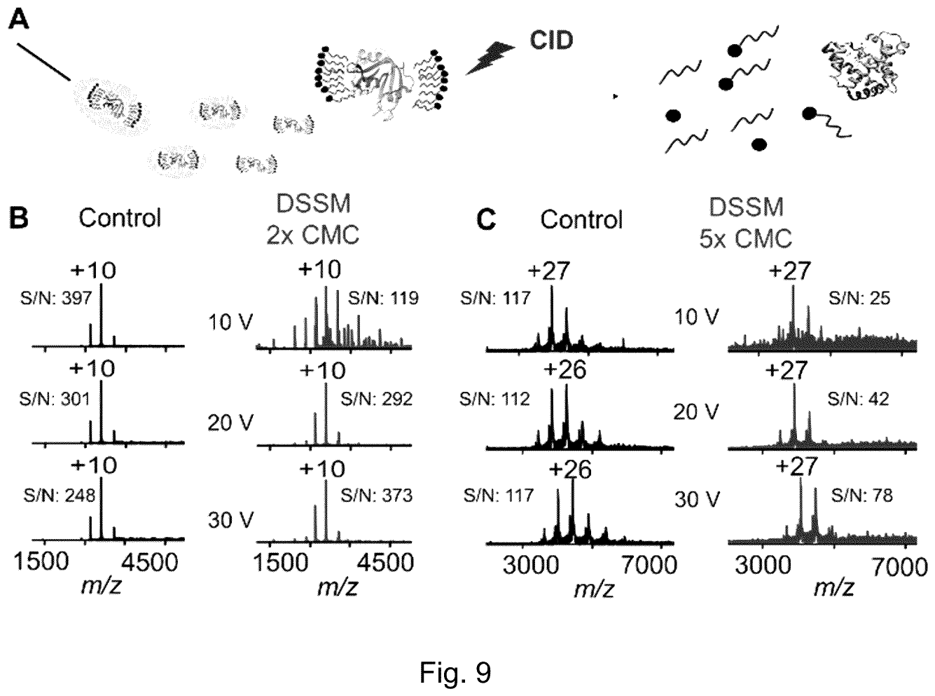

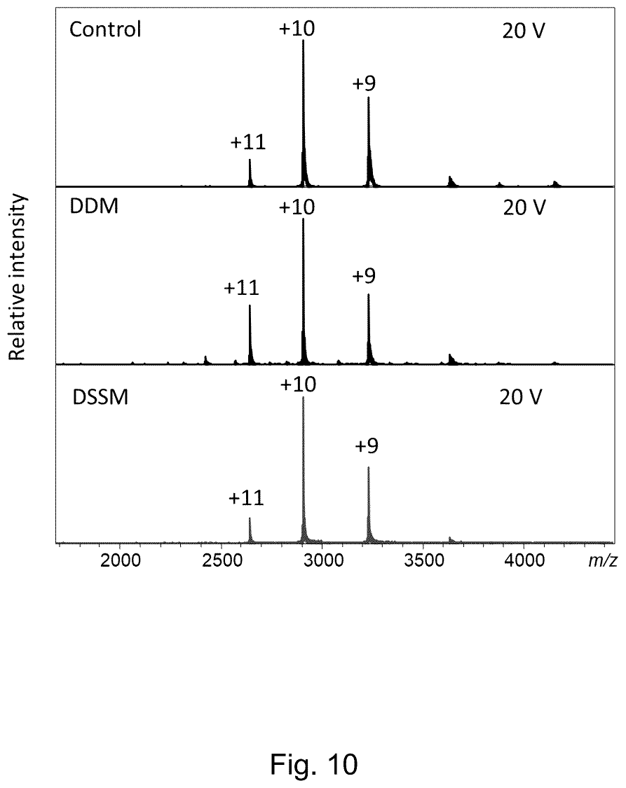

Next, it was tested if ion activation (i.e. collision-induced dissociation [CID]) could be used to dissociate the surfactant from the proteins and protein complexes for ESI-MS analysis under non-denaturing conditions 31 (FIG. 9). MS analysis of carbonic anhydrase (29.1 kDa) ammonium acetate with and without DSSM at a concentration of 2× CMC (critical micelle concentration) yielded spectra with significant signal suppression from DSSM monomers at lower collisional activation (2-10 V) (FIG. 9). When higher collisional activation (20-30 V) was applied, quality MS spectra were observed with a similar charge state distribution to the sample in ammonium acetate alone (FIG. 9). Similar results were observed for carbonic anhydrase in DDM at 2× CMC (FIG. 10).

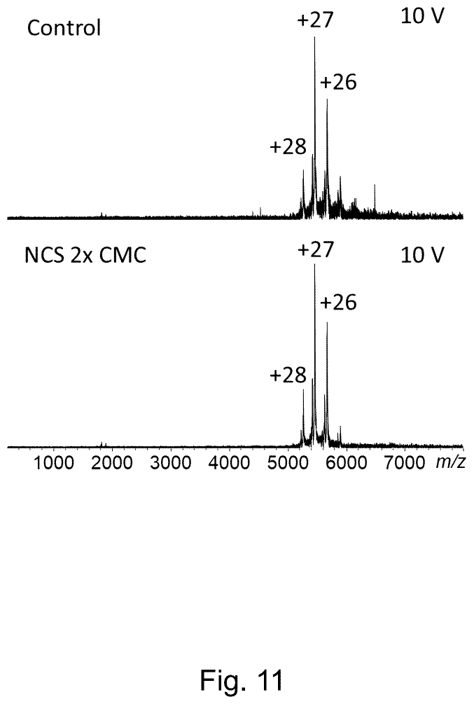

For the tetramer forming complex, alcohol dehydrogenase (147.5 kDa), quality spectra could be obtained at a low collisional voltage (10 V) with and without DSSM at 2× CMC (FIG. 11). DSSM was further tested at a higher concentration (5× CMC) using collisional activation energies of 20-30 V to achieve direct ESI-MS analysis (FIG. 9). Alternative activation methods, such as surface-induced dissociation (SID),⁴⁴ ultraviolet photodissociation (UVPD),^{45,46} infrared laser activation (IRMPD),¹⁸ may be implemented to remove DSSM in the gas phase for direct ESI-MS analysis.^{31,47,48}

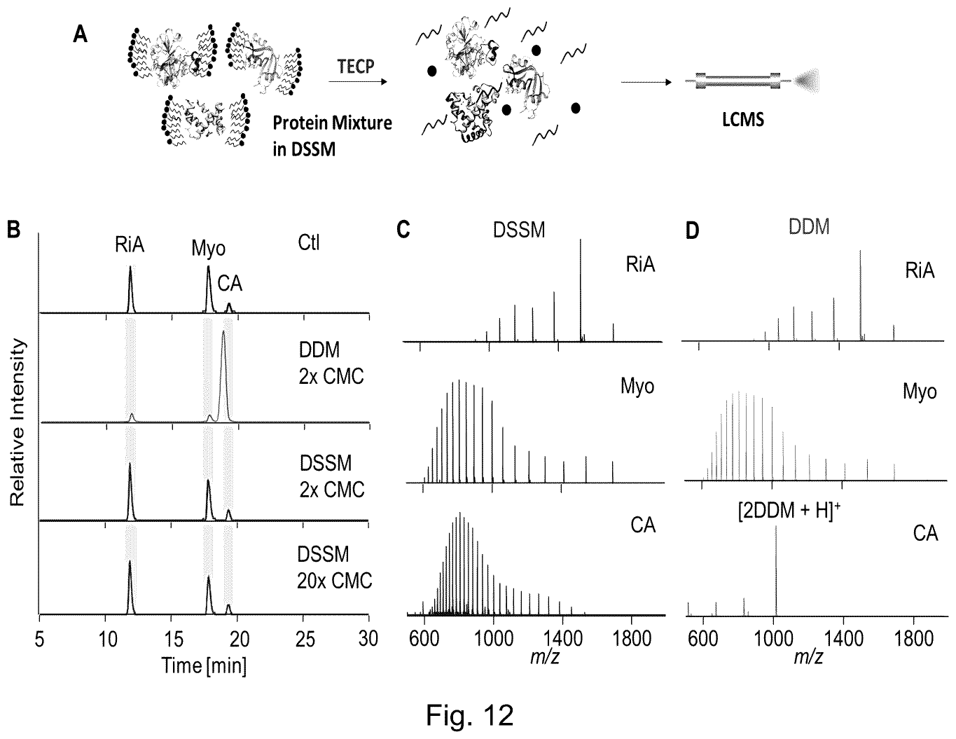

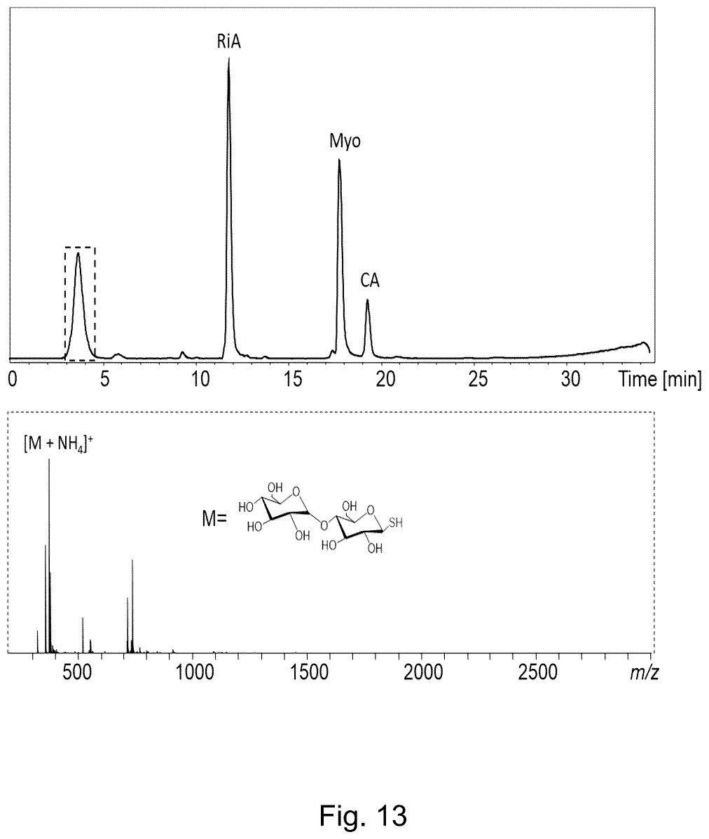

Evaluating DSSM LC-MS Compatible for Intact Protein Analysis. To evaluate the surfactant's compatibility with RPLC-MS, a mixture of standard proteins (ribonuclease A, myoglobin, and carbonic anhydrase) was analyzed with and without DSSM or DDM. DSSM did not influence the separation or the signal intensity of the standard proteins even at 20× CMC (FIG. 12). The improved compatibility with RPLC-MS compared to direct ESI-MS analysis results from the fact that the maltose head group after the degradation of DSSM elutes in the void volume before the proteins during LC separation (FIG. 13). Similarly, the addition of TCEP did not appear to have a deleterious effect. DDM, on the other hand, led to significant signal suppression in the chromatogram and mass spectra (FIG. 12). This demonstrates the promise of DSSM as a general replacement for nonionic surfactants like DDM for RPLC-MS applications.

DSSM-enabled Top-down Proteomics of Target Membrane Proteins. Membrane proteins represent an important class of drug targets but are generally difficult to study using top-down proteomics owing to their inherent insolubility outside the plasma membrane and low abundance.^{4, 33-36} Surfactants are the most common tool to extract, solubilize, and purify membrane proteins as they act as membrane mimics.^{7,38} Here, DSSM was assessed based on its compatibility with RPLC-MS to study membrane proteins, an important class of drug targets that are generally difficult to study using top-down proteomics owing to their inherent insolubility outside the plasma membrane and low abundance.^{33,35,36}

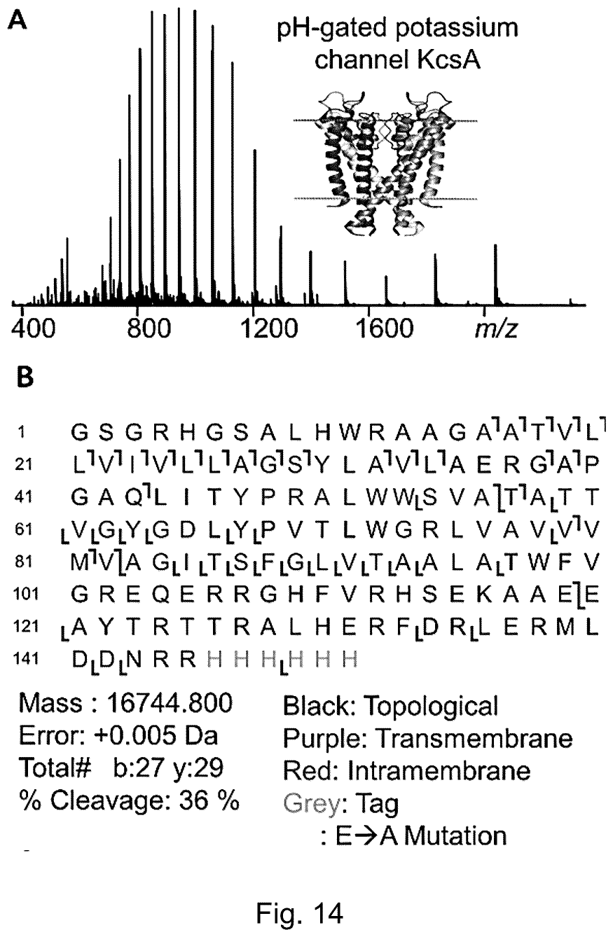

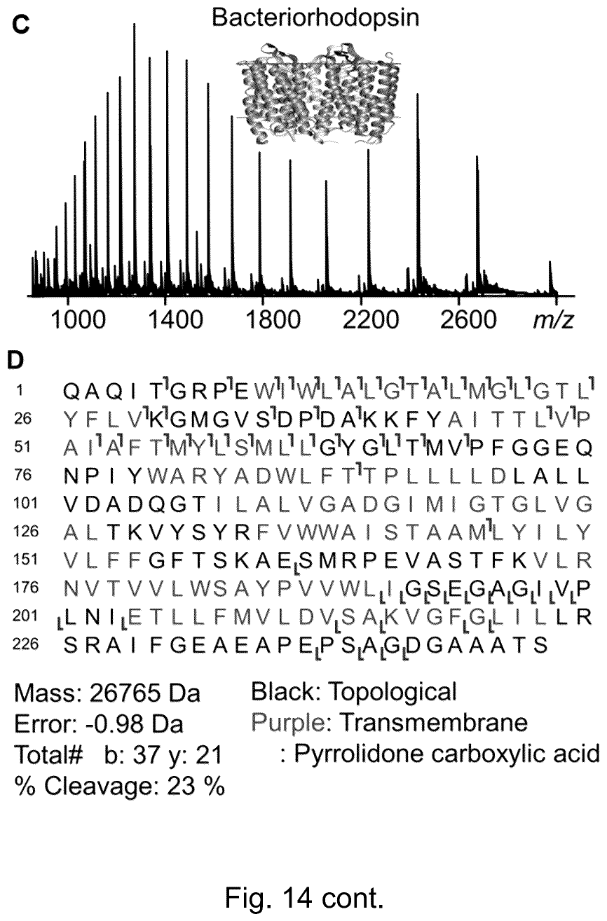

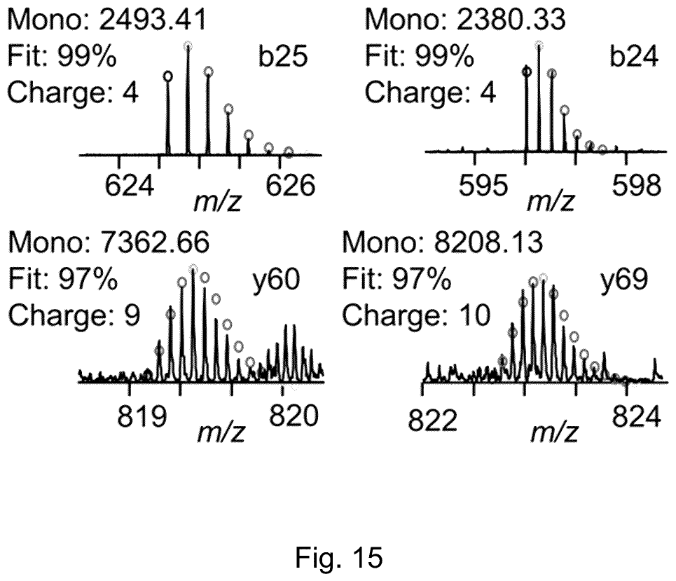

DSSM-aided membrane proteomic analysis was performed on a model ion channel protein, a pH-gated potassium channel (KcsA). After removing incompatible buffer components (salts, detergent, etc.) using a chloroform:methanol:water precipitation,²⁵ KcsA was solubilized in the DSSM (2× CMC). The surfactant was degraded with TCEP (in water or 50% isopropanol) and RPLC-MS/MS was performed using CID for fragmentation (FIG. 14, Panels A-B, and FIG. 15). Using MASH Explorer²⁶ for peak assignment and validation, good sequence coverage was observed on an LC-MS time scale with 27b ions and 29y ions representing 36% residue cleavage. Many of the bond cleavages were found in the transmembrane domains (TMD), in line with previous studies that characterized the fragmentation trends of intact integral membrane proteins.^{38,39} Furthermore, a mutation (E71A) that prevents channel inactivation⁴⁰ was successfully mapped (FIG. 14).

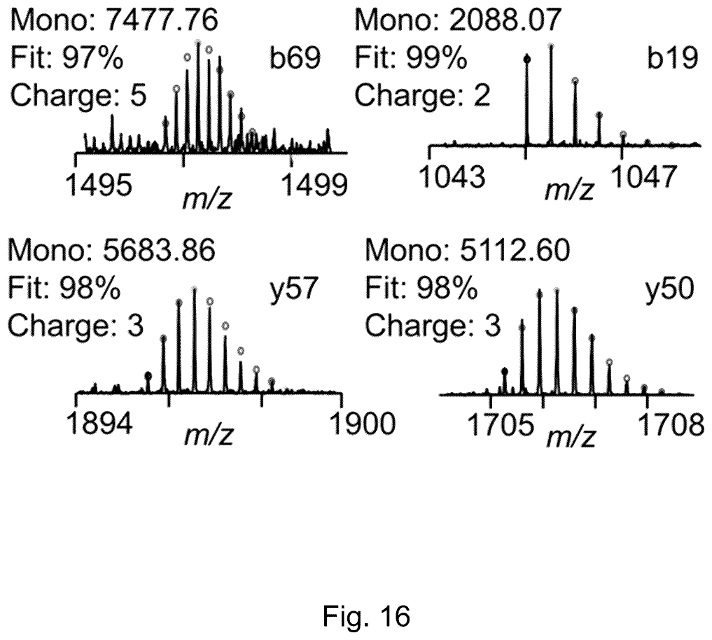

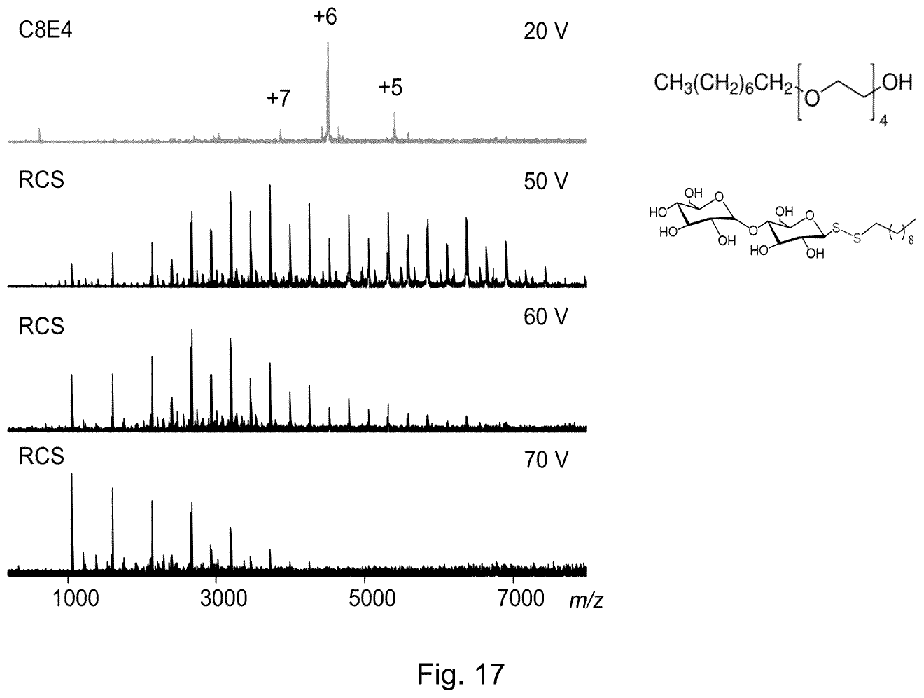

Furthermore, it was demonstrated that DSSM could enable the top-down analysis of bacteriorhodopsin, 41,42 a commercially available GPCR. After bacteriorhodopsin was solubilized in DSSM and degraded using TCEP (in water or 50% isopropanol), RPLC-MS/MS yielded 37b ions and 21y ions corresponding to 23% residue cleavage (FIG. 14, Panels C-D, and FIG. 16). A pyrrolidone carboxylic acid modification was localized to the N-terminus of the protein (FIG. 14, Panel D).

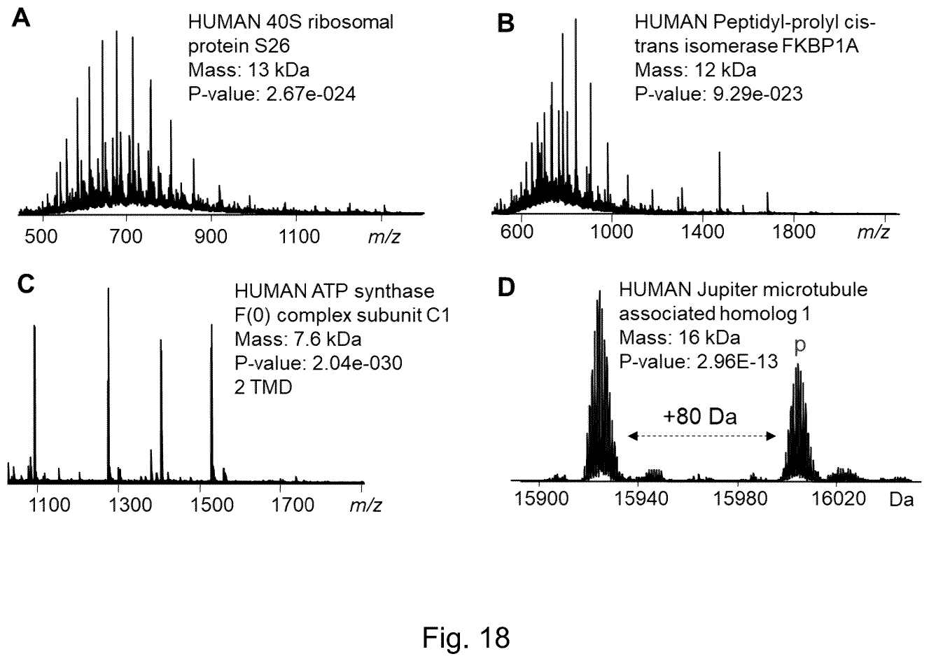

The use of DSSM was also evaluated for native MS of bacteriorhodopsin. Traditionally, the membrane protein-micelle complex is ionized and collisional activated to liberate the membrane protein in the gas phase. 31 It was hypothesized that the labile bond would facilitate easier surfactant dissociation. However, despite applying high collisional energy (70 eV) no signal could be seen for bacteriorhodopsin in DSSM (FIG. 17). Native MS signal could be obtained for bacteriorhodopsin in C8E4 micelles at relatively low collisional energies (20 eV). These results are consistent with the observation that membrane proteins in DDM micelles are difficult to remove in the gas phase without harsh activation conditions.^{36,43}

Top-down Proteomics of Endogenous Proteins using DSSM for Extraction. Surfactants are commonly used for extracting protein from cells and tissue for top-down proteomics.^{15, 20} For example, DDM was used to high sensitivity top-down proteomics using the nanoPOTs system.²² In this experiment, DSSM was used to extract endogenous protein from mammalian cells and directly analyzed using RPLC-MS/MS after surfactant degradation. Following TopPIC data analysis,²⁷ a total of 276 proteoforms⁶ were identified from 206 protein groups over four LC-MS/MS experiments (FIG. 18). Additionally, PTMs such as phosphorylation, methylation, and trimethylation were successfully localized using CID. Overall, it was demonstrated that DSSM is a valuable surfactant for cell lysis and enables proteoform identification using RPLC-MS/MS analysis

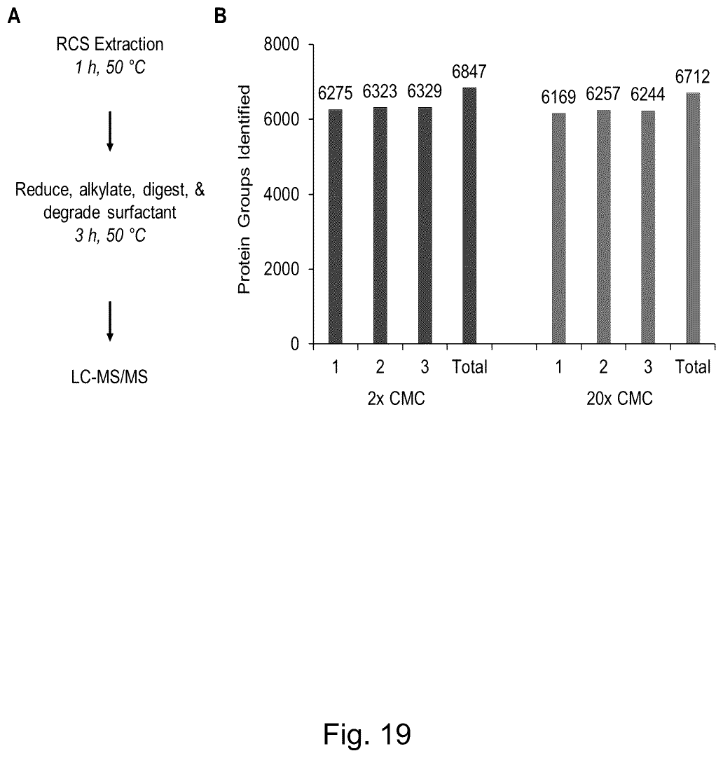

Single-pot NCS-aided Bottom-up Proteomics. In addition to their use in top-down proteomics, nonionic surfactants like DDM have become popular for shotgun proteomics, particularly in the context of high-sensitivity single-cell analysis.^{12,13} This is achievable for bottom-up proteomics because peptides elute before DDM, thereby avoiding the signal suppression observed with co-eluting intact proteins. However, there are still limits to the concentration of DDM that can be utilized and the potential loss of robustness caused by column fouling and clogging.¹³ Here, DSSM is used for a single-pot bottom-up proteomic approach as a degradable alternative to DDM (FIG. 19).

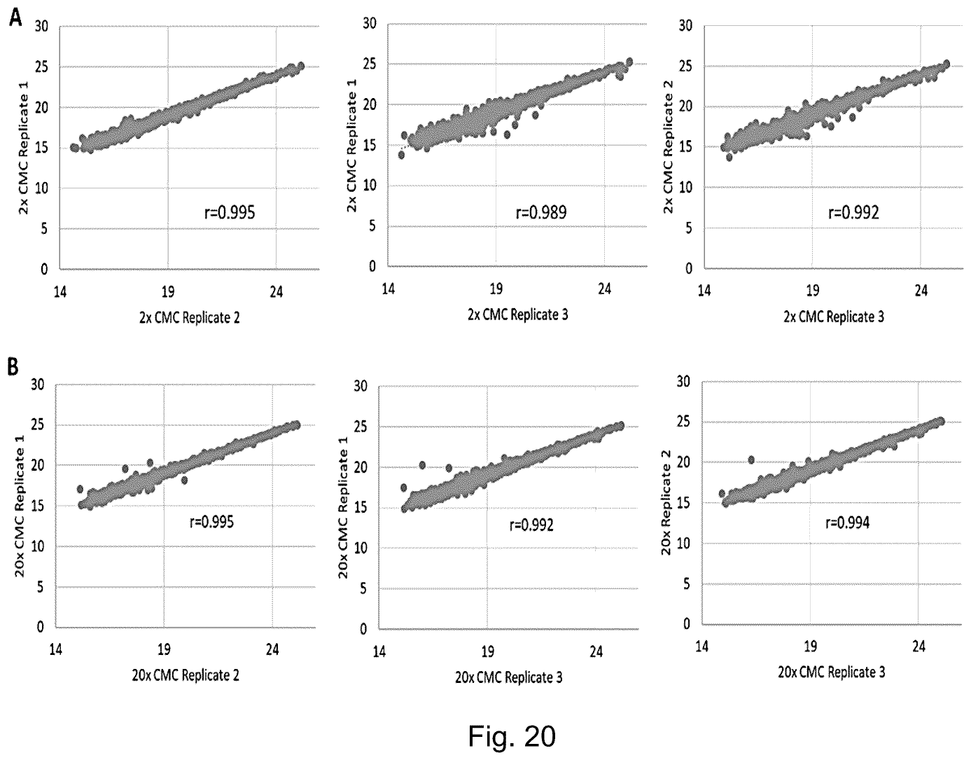

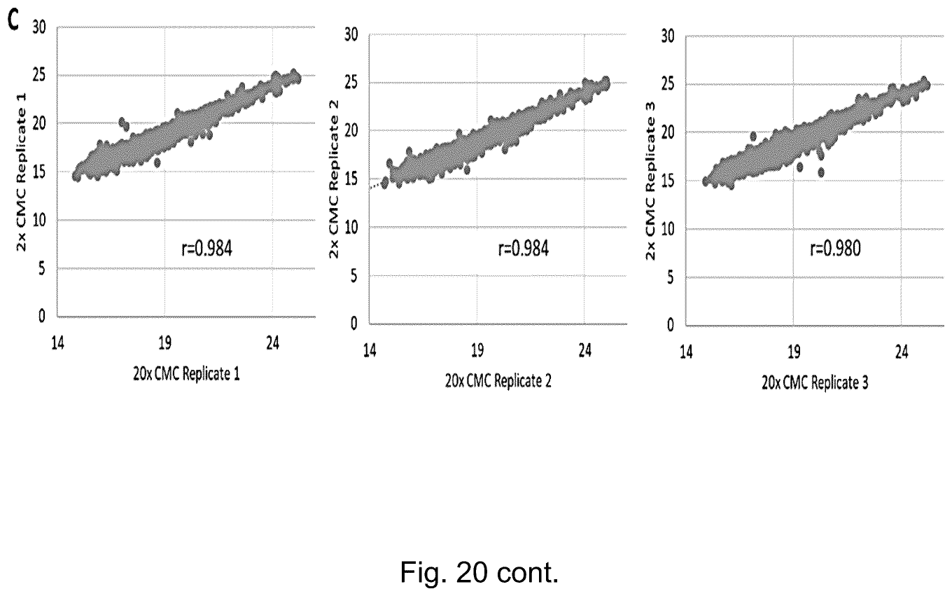

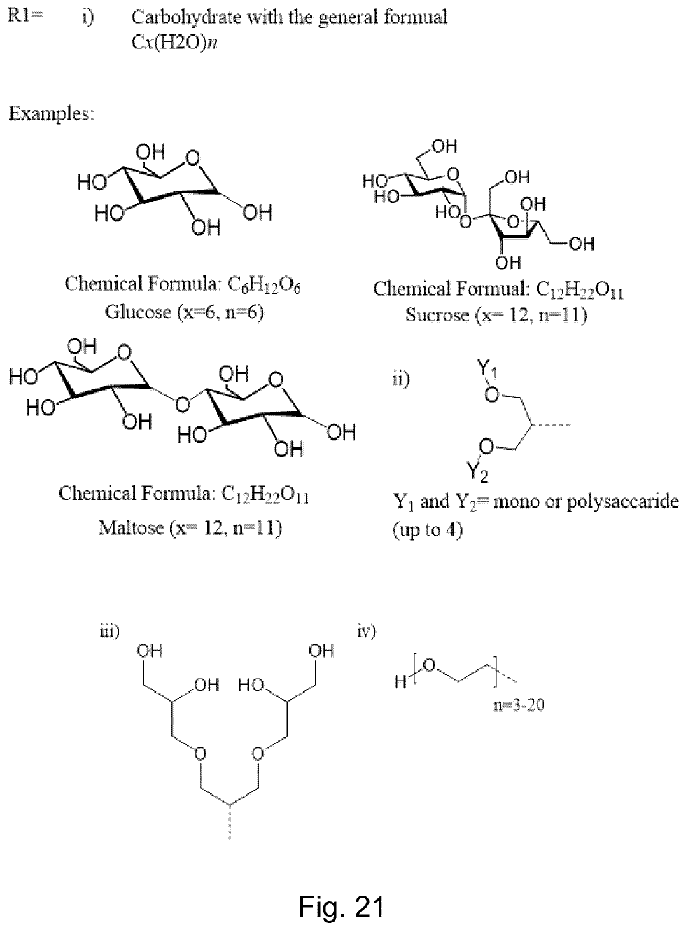

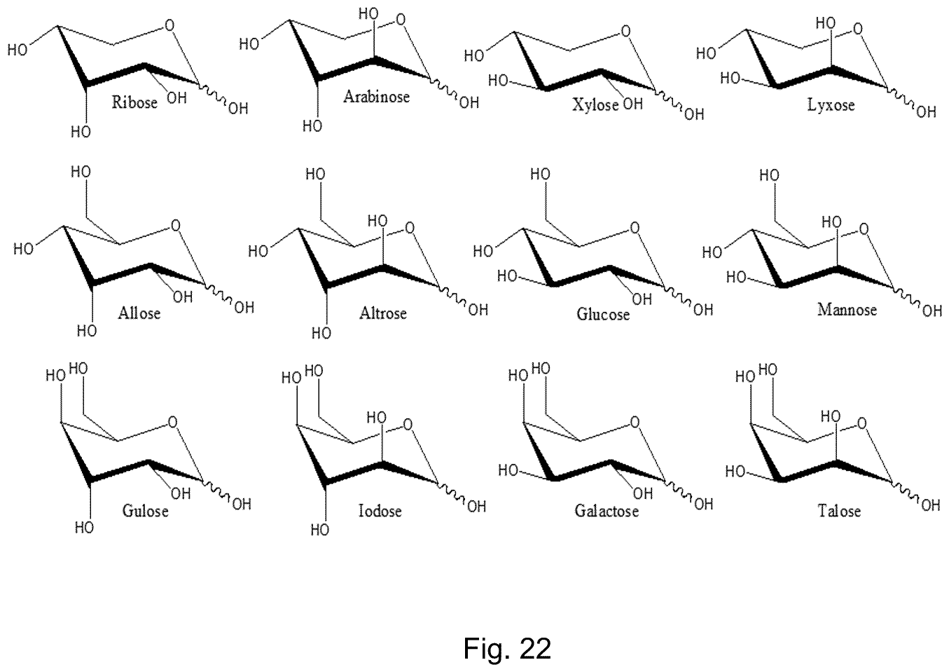

In this study, both 2× and 20× CMC of NCS were tested for cell lysis. Proteins were then reduced, alkylated, digested with trypsin, and the surfactant degraded in a single step. After a brief centrifugation step to remove cell debris, RPLC-MS/MS analysis was performed identifying 6847 and 6712 protein groups for 2× and 20× CMC respectively. Moreover, excellent reproducibility was observed for quantifying protein abundance across extraction replicates with a Pearson correlation coefficient of 0.992±0.002 (average±standard deviation) and 0.994±0.001 for 2× and 20× CMC respectively (FIG. 20). Additionally, a Pearson correlation coefficient of 0.983±0.002 was found when comparing 2× and 20× indicating there was little difference between the extraction conditions. In summary, the NCS-aided approach is a highly quantitative approach for bottom-up analysis of thousands of proteins from cell lysate.

In summary, this example shows for the first time the use of NCS for proteomics. n-Decyl-disulfide-β-D-maltoside (DSSM) is a cleavable DDM mimic and is directly compatible with direct ESI-MS analysis of intact proteins and top-down proteomics. DSSM was generally compatible with ESI-MS as well as RPLC-MS analysis circumventing the characteristic signal suppression typically observed for surfactants. It was demonstrated that DSSM enables the top-down proteomic characterization of a model ion channel (KcsA), GPCR (bacteriorhodopsin), and endogenous proteins extracted from cell lysates. DSSM and other NCS represent important and versatile surfactants that can facilitate protein sample preparation under non-denaturing conditions for a myriad of proteomic and structural biology applications and acts as a general replacement for DDM.

### Example 2

Synthesis of DSSM.

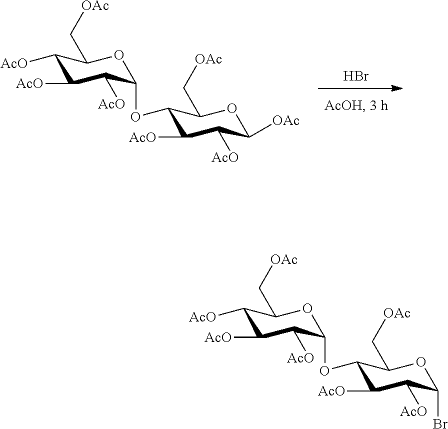

β-D-maltose octaacetate (3.39 g, 5.00 mmol, 1.00 equiv.) was added into a dray flask with 15 mL DCM, and 15 mL 33% wt. HBr in AcOH. Nitrogen gas was bubbled into the reaction mixture for 10 min, and the reaction was allowed to proceed at room temperature under nitrogen gas for 3 h. The mixture was then partitioned between 40 mL H₂O and 40 mL DCM, and the aqueous layer was re-extracted with DCM (3×20 mL). The organic layers were then combined and neutralized with saturated NaHCO₃, followed by a brine wash (80 mL). The organic layer was then concentrated to an amorphous solid in vacuo, and the resulting crude was used in the next step without further purification.

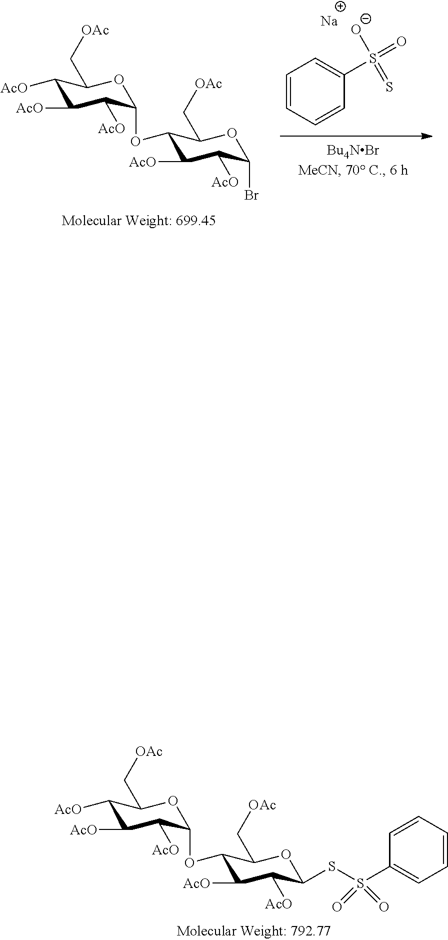

A dry flask was charged with sodium benzenethiosulfonate (561 mg, 2.86 mmol, 2 equiv.), tetrabutylammonium bromide (46.1 mg, 0.143 mmol, 0.1 equiv), and peracetylated α-maltosylbromide (1.00 g, 1.43 mmol, 1.00 equiv). The flask was capped and flushed with nitrogen before adding anhydrous acetonitrile (40 mL). The reaction was then heated to 70° C. and allowed to proceed with stirring under inert conditions for 6 h. Solvent was removed in vacuo and the residue was partitioned between EtOAc (40 mL) and water (40 mL). The aqueous layer was extracted twice more with EtOAc (40 mL). The organic layers were combined, washed once with brine (40 mL), and dried with MgSO₄. The solvent was removed in vacuo, and the crude solid was purified by silica flash chromatography (50/50 hexanes/EtOAc, R_{f}=0.2, α anomer elutes first) to yield the β-maltosyl phenylthiosulfonate as a white solid. Yield 464 mg, 40%.

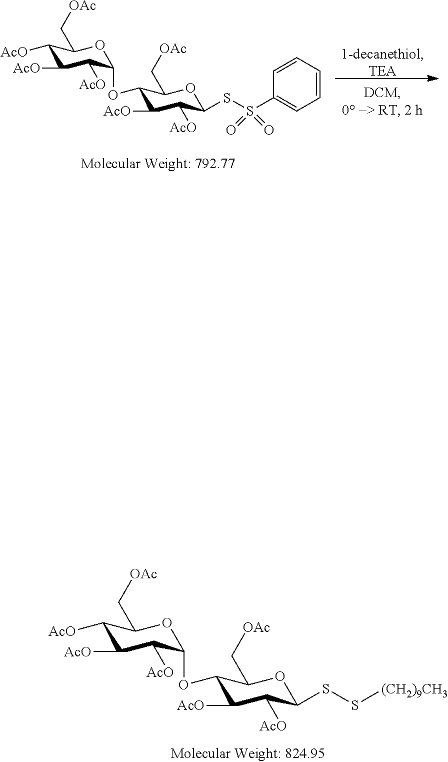

The β-maltosyl phenylthiosulfonate (0.126 mmol, 100 mg, 1 equiv.) from step 2 was dissolved in CH₂Cl₂ (1 mL) and TEA (35 μL, 0.252 mmol, 2 equiv.). This mixture was allowed to cool to 0° C. before adding a solution of cold 1-decanethiol (52 μL, 0.252 mmol, 2 equiv.) in CH₂Cl₂ (1 mL) dropwise over 15 min. The reaction mixture was allowed to warm to room temperature and stir for 2 h. The solvent was removed in vacuo and the residue was purified by silica flash chromatography (50/50 hexanes/EtOAc, R_{f}=0.55) to yield the desired disulfide as a white solid. Yield 83 mg, 80%.

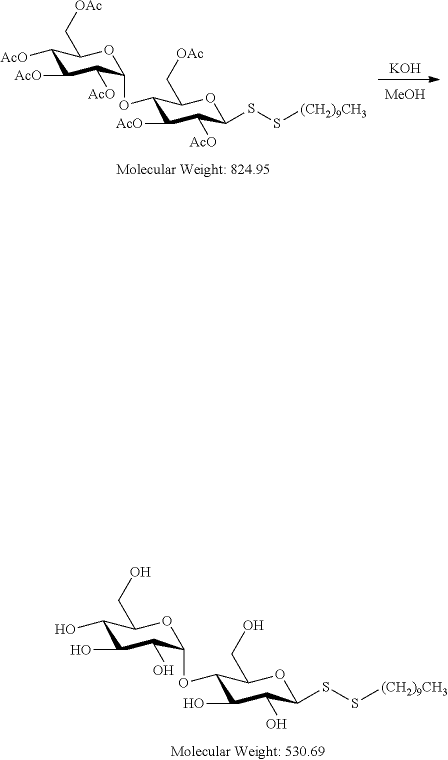

The disulfide from step 3 (25 mg) was dissolved in MeOH (250 μL), to which a solution of KOH in MeOH (1% wt., 500 μL) was added. The reaction was allowed to proceed for 30 min and completion was verified by TLC. To the reaction was added Dowex until pH=7 was reached. The mixture was then filtered, concentrated to 200 μL, diluted with 800 μL DCM, and purified by silica flash chromatography (80/20 CH₂Cl₂/MeOH, R_{f}=0.5) to yield pure CCD-2 as a white solid. Yield 87%.

Purification of KcsA. Gene fragment of KcsA(E71A) with a C-terminal His-tag and codon optimization was purchased from Twist Bioscience and cloned into the C-terminus of a chimeric hybrid protein derived from E. coli maltose-binding protein and Saposin A in pETDuet1 plasmid, with a thrombin cleavage site placed between hybrid protein and KcsA. The construct was transformed into E. coli SHuffle cells, grown at 37° C. in 2XYT with 100 μg/L ampicillin. At OD600=1.0, overexpression was induced with 1 mM IPTG at 16° C. overnight.

After cells were harvested, cells were lysed in 50 mM tris-HCl, 100 mM KCl, 1 mM PMSF, and 0.5% w/v DDM. The fusion protein was purified over HisPrep FF column, and eluted with 500 mM imidazole, followed by dialysis against 100 volume of 50 mM tris-HCl, 100 mM KCI. The fusion protein was cleaved by thrombin immobilized on agarose beads (Sigma-Aldrich) overnight at 10° C. The cleavage product was purified using a Ni column. The protein was mixed with 10 mM DDM, and further purified over a Superdex 200 column in 150 mM ammonium acetate pH 7.4.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

When a group of materials, compositions, components, or compounds is disclosed herein, it is understood that all individual members of those groups and all subgroups thereof are disclosed separately. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. Additionally, the end points in a given range are to be included within the range. In the disclosure and the claims, “and/or” means additionally or alternatively. Moreover, any use of a term in the singular also encompasses plural forms.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements.

One of ordinary skill in the art will appreciate that starting materials, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations which is not specifically disclosed herein. Headings are used herein for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

### REFERENCES

1. R. Aebersold et al., Nat Chem Biol, 2018, 14, 206-214.

2. R. Aebersold and M. Mann, Nature, 2003, 422, 198-207.

3. B. T. Chait, Science, 2006, 314, 65.

4. K. A. Brown, J. A. Melby, D. S. Roberts and Y. Ge, Expert Rev Proteomics, 2020, 1-15.

5. B. Chen, K. A. Brown, Z. Lin and Y. Ge, Analytical Chemistry, 2018, 90, 110-127.

6. L. M. Smith and N. L. Kelleher, Nat Methods, 2013, 10, 186-187.

7. A. E. Speers and C. C. Wu, Chem Rev, 2007, 107, 3687-3714.

8. R. R. Loo, N. Dales and P. C. Andrews, Protein Sci, 1994, 3, 1975-1983.

9. J. R. Deschamps, Journal of Liquid Chromatography, 1986, 9, 1635-1653.

10. J. R. Wisniewski, A. Zougman, N. Nagaraj and M. Mann, Nat Methods, 2009, 6, 359-362.

11. M. HaileMariam, R. V. Eguez, H. Singh, S. Bekele, G. Ameni, R. Pieper and Y. Yu, Journal of Proteome Research, 2018, 17, 2917-2924.

12. X. Zhang, Molecular & cellular proteomics: MCP, 2015, 14, 2441-2453.

13. C. F. Tsai et al., Communications Biology, 2021, 4, 265.

14. D. P. Donnelly et al., Nat Methods, 2019, 16, 587-594.

15. C. Kachuk and A. A. Doucette, Journal of Proteomics, 2018, 175, 75-86.

16. E. I. Chen, D. Cociorva, J. L. Norris and J. R. Yates, Journal of proteome research, 2007, 6, 2529-2538.

17. Y. Q. Yu, M. Gilar, P. J. Lee, E. S. Bouvier and J. C. Gebler, Anal Chem, 2003, 75, 6023-6028.

18. Y. H. Chang, Z. R. Gregorich, A. J. Chen, L. Hwang, H. Guner, D. Yu, J. Zhang and Y. Ge, Journal of Proteome Research, 2015, 14, 1587-1599.

19. S. V. Saveliev, C. C. Woodroofe, G. Sabat, C. M. Adams, D. Klaubert, K. Wood and M. Urh, Anal Chem, 2013, 85, 907-914.

20. K. A. Brown, B. Chen, T. M. Guardado-Alvarez, Z. Lin, L. Hwang, S. Ayaz-Guner, S. Jin and Y. Ge, Nature Methods, 2019, 16, 417-420.

21. K. Brown, T. Tucholski, C. Eken, S. Knott, Y. Zhu, S. Jin and Y. Ge, Angewandte Chemie International Edition, 2020, 59.

22. M. Zhou, N. Uwugiaren, S. M. Williams, R. J. Moore, R. Zhao, D. Goodlett, I. Dapic, L. Pas̆a-Tolić and Y. Zhu, Analytical Chemistry, 2020, 92, 7087-7095.

23. M. Zhou, N. Uwugiaren, S. M. Williams, R. J. Moore, R. Zhao, D. Goodlett, I. Dapic, L. Pas̆a-Tolić and Y. Zhu, Anal Chem, 2020, 92, 7087-7095.

24. D. Xue, J. Wang, X. Song, W. Wang, T. Hu, L. Ye, Y. Liu, Q. Zhou, F. Zhou, Z. X. Jiang, Z.-J. Liu and H. Tao, Langmuir, 2019, 35, 4319-4327.

25. J. Folch, I. Ascoli, M. Lees, J. A. Meath and B. N. Le, J Biol Chem, 1951, 191, 833-841.

26. Z. Wu, D. S. Roberts, J. A. Melby, K. Wenger, M. Wetzel, Y. Gu, S. G. Ramanathan, E. F. Bayne, X. Liu, R. Sun, I. M. Ong, S. J. Mcllwain and Y. Ge, Journal of Proteome Research, 2020, 19, 3867-3876.

27. Q. Kou, L. Xun and X. Liu, Bioinformatics, 2016, 32, 3495-3497.

28. The UniProt Consortium, Nucleic acids research, 2017, 45, D158-D169.

29. F. Meier, A. D. Brunner, S. Koch, H. Koch, M. Lubeck, M. Krause, N. Goedecke, J. Decker, T. Kosinski, M. A. Park, N. Bache, O. Hoerning, J. Cox, O. Rather and M. Mann, Mol Cell Proteomics, 2018, 17, 2534-2545.

30. F. Yu, S. E. Haynes, G. C. Teo, D. M. Avtonomov, D. A. Polasky and A. I. Nesvizhskii, Mol Cell Proteomics, 2020, 19, 1575-1585.

31. A. Laganowsky, E. Reading, J. T. Hopper and C. V. Robinson, Nat Protoc, 2013, 8, 639-651.

32. D. S. Chorev, L. A. Baker, D. Wu, V. Beilsten-Edmands, S. L. Rouse, T. Zeev-Ben-Mordehai, C. Jiko, F. Samsudin, C. Genie, S. Khalid, A. G. Stewart, S. J. Matthews, K. Grünewald and C. V. Robinson, Science, 2018, 362, 829.

33. A. O. Helbig, A. J. Heck and M. Slijper, J Proteomics, 2010, 73, 868-878.

34. N. P. Barrera and C. V. Robinson, Annu Rev Biochem, 2011, 80, 247-271.

35. K. A. Brown, T. Tucholski, A. J. Alpert, C. Eken, L. Wesemann, A. Kyrvasilis, S. Jin and Y. Ge, Analytical Chemistry, 2020, 92, 15726-15735.

36. J. E. Keener, G. Zhang and M. T. Marty, Analytical Chemistry, 2021, 93, 583-597.

37. H. Hussain, Y. Du, N. J. Scull, J. S. Mortensen, J. Tarrasch, H. E. Bae, C. J. Loland, B. Byrne, B. K. Kobilka and P. S. Chae, Chemistry (Weinheim an der Bergstrasse, Germany), 2016, 22, 7068-7073.

38. O. S. Skinner, A. D. Catherman, B. P. Early, P. M. Thomas, P. D. Compton and N. L. Kelleher, Anal Chem, 2014, 86, 4627-4634.

39. W. Cohn, R. Huguet, V. Zabrouskov and J. Whitelegge, J Am Soc Mass Spectrom, 2021, 32, 1380-1387.

40. E. Vales and M. Raja, J Membr Biol, 2010, 234, 1-11.

41. J. P. Whitelegge, C. B. Gundersen and K. F. Faull, Protein science: a publication of the Protein Society, 1998, 7, 1423-1430.

42. C. M. Ryan, P. Souda, S. Bassilian, R. Ujwal, J. Zhang, J. Abramson, P. Ping, A. Durazo, J. U. Bowie, S. S. Hasan, D. Baniulis, W. A. Cramer, K. F. Faull and J. P. Whitelegge, Molecular & cellular proteomics: MCP, 2010, 9, 791-803.

43. E. Reading, I. Liko, T. M. Allison, J. L. Benesch, A. Laganowsky and C. V. Robinson, Angew Chem lnt Ed Engl, 2015, 54, 4577-4581.

44. Snyder, D. T., Panczyk, E. M., Somogyi, A., Kaplan, D. A. & Wysocki, V. Anal Chem, 2020, 92, 11195-11203.

45. Greisch, J. F. et al., Chem Sci, 2019, 10, 7163-7171.

46. Crittenden, C. M. et al., Journal of the American Society for Mass Spectrometry, 2020, 31, 1140-1150.

47. Mikhailov, V. A. et al., Anal Chem, 2016, 88, 7060-7067.

48. Calabrese, A. N., Watkinson, T. G., Henderson, P. J. F., Radford, S. E. & Ashcroft, A. E., Anal Chemistry, 2015, 87, 1118-1126.

1. A chemically-cleavable surfactant comprising: a) hydrophilic head, b) a hydrophobic tail, and c) a cleavable disulfide bond covalently linking the hydrophilic head and hydrophobic tail, where the cleavable disulfide bond is able to release the hydrophilic head, release the hydrophobic tail, and/or break apart upon exposure to a reducing agent.

2. The chemically-cleavable surfactant of claim 1, wherein the reducing agent is selected from the group consisting of tris(2-carboxyethyl)phosphine (TCEP), dithiothreitol (DTT), beta mercaptoethanol (BME), dithiobutylamine (DTBA), [N,N′-dimethyl-N,N′-bis(mercaptoacetyl) hydrazine (DMH), glutathione (GSH), borane dimethylsulfide (BMS), cysteine-HCl, 2-mercaptoethylamine-HCl, and combinations thereof.

3. The chemically-cleavable surfactant of claim 1, wherein the chemically-cleavable surfactant has the formula: 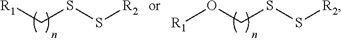
wherein R1 is a hydrophilic moiety and R2 is a hydrophobic moiety, and n is an integer selected from 1 to 6.

4. The chemically-cleavable surfactant of claim 3, wherein R1 comprises one or more branched or unbranched monosaccharides, disaccharides, polysaccharides, or combinations thereof.

5. The chemically-cleavable surfactant of claim 3, wherein R1 comprises a triglycerol, tetraglycerol, pentaglycerol, hexaglycerol, a decaglycerol, or combinations thereof.

6. The chemically-cleavable surfactant of claim 3, wherein R1 comprises an oligoglycerol having the formula:

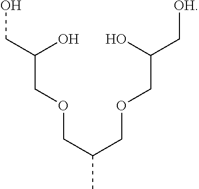

7. The chemically-cleavable surfactant of claim 3, wherein R1 comprises an polyethylene glycol having the formula: 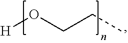
wherein n is an integer selected from 3-20.

8. The chemically-cleavable surfactant of claim 3, wherein R1 comprises a group having the formula: 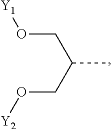
where Y1 and Y2, independently form one another, are each a monosaccharide, disaccharide, or polysaccharide.

9. The chemically-cleavable surfactant of claim 3, wherein R2 is a substituted or unsubstituted alkyl, alkenyl, alkynyl, or aryl group having between 2 and 16 carbon atoms.

10. The chemically-cleavable surfactant of claim 3, wherein R2 is an alkyl group the formula: 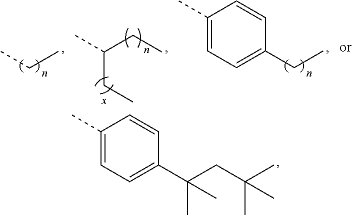
where n is an integer selected from 1-30.

11. The chemically-cleavable surfactant of claim 1, where the chemically-cleavable surfactant of the method has the formula:

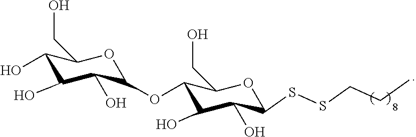

12. A method of solubilizing a compound comprising the steps of:
a) mixing the compound with a chemically-cleavable surfactant in a solution until the compound is dissolved in the solution;
b) exposing the solution containing the chemically-cleavable surfactant and dissolved compound to a reducing agent, thereby cleaving the chemically-cleavable surfactant, wherein the chemically-cleavable surfactant comprises: i) hydrophilic head, ii) a hydrophobic tail, and iii) a cleavable disulfide bond covalently linking the hydrophilic head and hydrophobic tail, where the cleavable disulfide bond is able to release the hydrophilic head, release the hydrophobic tail, and/or break apart upon exposure to the reducing agent.

13. The method of claim 12, wherein the reducing agent is selected from the group consisting of tris(2-carboxyethyl)phosphine (TCEP), dithiothreitol (DTT), beta mercaptoethanol (BME), dithiobutylamine (DTBA), [N,N′-dimethyl-N,N′-bis(mercaptoacetyl) hydrazine (DMH), and combinations thereof.

14. The method of claim 12, wherein the reducing agent is tris(2-carboxyethyl)phosphine (TCEP).

15. The method of claim 12 further comprising performing a chromatography step on the exposed solution.

16. A method for analyzing a compound comprising the steps of:
a) mixing the compound with a chemically-cleavable surfactant in a solution until the compound is dissolved in the solution, wherein the chemically-cleavable surfactant comprises: i) a hydrophilic head, ii) a hydrophobic tail, and iii) a cleavable disulfide bond covalently linking the hydrophilic head and hydrophobic tail, where the cleavable disulfide bond is able to release the hydrophilic head, release the hydrophobic tail, and/or break apart upon exposure to collisional energy during mass spectrometry;
b) exposing the solution containing the chemically-cleavable surfactant and dissolved compound to collisional energy or electron based fragmentation within a mass spectrometer device, thereby decomposing the chemically-cleavable surfactant; and
c) performing mass spectrometry (MS) analysis on a portion of the exposed solution containing the compound.

17. The method of claim 16, wherein exposing the solution containing the chemically-cleavable surfactant and dissolved compound to collisional energy comprises performing collision-induced dissociation (CID).

18. The method of claim 16, wherein the compound comprises one or more polypeptides, intact soluble protein complexes, or combinations thereof.

19. The method of claims 16, wherein the chemically-cleavable surfactant of the method has the formula: 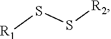
wherein, R1 is a hydrophilic moiety comprising between 1 and 16 carbon atoms, and R2 is a hydrophobic moiety comprising a substituted or unsubstituted alkyl, alkenyl, alkynyl, or aryl group having between 1 and 16 carbon atoms.

20. The method of claim 19, wherein R1 comprises one or more branched or unbranched monosaccharides, disaccharides, polysaccharides, or combinations thereof.

21. The method of claim 19, wherein R1 comprises a group having the formula: 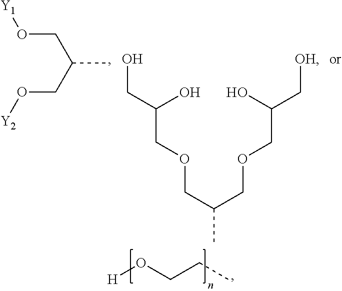
where n is an integer selected from 3-20, and Y1 and Y2, independently form one another, are each a monosaccharide, disaccharide, or polysaccharide.

22. The method of claim 16, wherein the chemically-cleavable surfactant of the method has the formula: [chemical structure]


SEQUENCE LISTING (2 sequences)

SEQ ID NO: 1 - protein, 151 aa, synthetic construct
GSGRHGSALHWRAAGAATVLLVIVLLAGSYLAVLAERGAPGAQLITYPRALWWSVATATTVGYGDLYPVTLWGRLVAVVVMVAGITSFGLVTAALATWFVGREQERRGHFVRHSEKAAEEAYTRTTRALHERFDRLERMLDDNRRHHHHHH

SEQ ID NO: 2 - protein, 248 aa, synthetic construct
QAQITGRPEWIWLALGTALMGLGTLYFLVKGMGVSDPDAKKFYAITTLVPAIAFTMYLSMLLGYGLTMVPFGGEQNPIYWARYADWLFTTPLLLLDLALLVDADQGTILALVGADGIMIGTGLVGALTKVYSYRFVWWAISTAAMLYILYVLFFGFTSKAESMRPEVASTFKVLRNVTVVLWSAYPVVWLIGSEGAGIVPLNIETLLFMVLDVSAKVGFGLILLRSRAIFGEAEAPEPSAGDGAAATS